(19) United States
(12) Patent Application Publication
(10) Pub. No.: US 2003/0073069 A1
(43) Pub. Date: Apr. 17, 2003

(54) Drug rescue by redesign of ADMET/PK properties

(76) Inventors: Harold E. Selick, Belmont, CA (US); Kenneth R. Korzekwa, Mountain View, CA (US); Katrin Mackarehtschian, Redwood City, CA (US)

Correspondence Address: BEYER WEAVER & THOMAS LLP, P.O. BOX 778, BERKELEY, CA, 94704-0778, US

(21) Appl. No.: 09/978,671

(22) Filed: Oct. 15, 2001

(51) Int. Cl. C12Q 1/00; G06G 7/48; G06G 7/58; G06F 19/00; G01N 33/48; G01N 33/50

(52) U.S. Cl. 435/4; 702/19; 703/11

(57) ABSTRACT

Otherwise efficacious drugs having an ADMET/PK (absorption, distribution, metabolism, elimination, toxicity, i.e., pharmacokinetic) problem are redesigned or “rescued” by applying computational techniques that identify related chemical structures that preserve the initial drug's effectiveness but improve its ADMET/PK properties. The otherwise efficacious drug may be subjected to a suite of computational tools that identify sites responsible for problematic ADMET/PK properties and/or identify related compounds that have improved ADMET/PK properties.

### CROSS-REFERENCE TO RELATED APPLICATIONS

This patent application is related to U.S. patent application Ser. No. 09/368,511, “Use of Computational and Experimental Data to Model Organic Compound Reactivity in Cytochrome p450 Mediated Reactions and to Optimize the Design of Pharmaceuticals,” filed Aug. 5, 1999 by Korzekwa et al. (Atty Docket No.: CAMIP001), U.S. patent application Ser. No. 09/613,875, “Relative Rates of Cytochrome p450 Metabolism,” filed Jul. 10, 2000 by Korzekwa et al. (Atty Docket No.: CAMIP002), U.S. Provisional Patent Application No. 60/217,227, “Accessibility Correction Factors for Quantum Mechanical and Molecular Models of Cytochrome p450 Metabolism,” filed Jul. 10, 2000 by Ewing et al. (Atty Docket No.: CAMIP004P); U.S. patent application Ser. No. 09/902,470, “Accessibility Correction Factors For Electronic Models Of Cytochrome P450 Metabolism,” filed Jul. 9, 2001 by Korzekwa et al. (CAMIP004); and U.S. patent application Ser. No. 09/811,283, “Predicting Metabolic Stability of Drug Molecules,” filed Mar. 15, 2001 by Ewing et al. (Atty Docket No.: CAMIP005). These patent applications, as well as any other patents, patent applications and publications cited herein, are hereby incorporated by reference in their entirety for all purposes.

### FIELD OF THE INVENTION

The present invention relates generally to systems and methods for redesigning or “rescuing” ethical pharmaceutical drugs, withdrawn drugs, lead compounds and other compounds. More specifically, the invention relates to systems and methods for structurally redesigning such compounds in order to improve one or more of their ADMET/PK (absorption, distribution, metabolism, elimination, toxicity, i.e., pharmacokinetic) properties.

### BACKGROUND OF THE INVENTION

Drug development is an extremely expensive and lengthy process. The cost of bringing a single drug to market is about $500 million to $1 billion dollars, with the development time being about 8 to 15 years. Drug development typically involves the identification of 1000 to 100,000 candidate compounds distributed across several

Once a drug has passed FDA trials and is offered on the market as an ethical pharmaceutical drug, the ADMET/PK properties may not be ideal. The drug may continue to be used, despite its ADMET/PK problems, or it may be withdrawn from use when a latent ADMET/PK problem is discovered. Another serious is that the drug may have adverse interactions with othe compounds. One compound may induce or inhibit the metabolism, absorption or tissue distribution of anothe compound, thus altering its efficacy and sometimes increasing its toxicity to dangerous levels.

In view of the foregoing, it would be beneficial if ethically-used drugs, withdrawn drugs and lead compounds could be quickly and precisely redesigned to improve their ASMET/PK properties. Such a technique could “rescue” drugs that are undesiraable because of their ADMET/PK properties, thus preventing the enormous cost of developing the drug in the first place frim going to waste.

### SUMMARY OF THE INVENTION

The present invention relates generally to systems and methods for redesigning or “rescuing” ethical pharmaccutical drugs, withdrawn drugs, lead compounds and other compounds. More specifically, the invention relates to systems and methods for ADMET/PK (absorption, distribution, metabolism, elimination, toxicity, i.e., pharmacokinetic) properties.

One aspect of the invention pertains to methods for redesigning a therapeutic chemical compound to improve at least one of its ADMET/PK properties, the method including the operations of identifying a therapeutic chemical compound that is a drug candidate, drug or withdrawn drug, analyzing the structure of the therapeutic chemical compound with a model that predicts one or more ADMET/PK properties of chemical compounds based upon their chemical structures, mext, from an analysis of the therapeutic chemical compound with the model, identifying one or more structural features of the therapeutic chemical compound that can be improved, and specifying a compound to possess an ADMET/PK property that can be improved, and specifying a modification for the one or more structural features to produce a modified chemical compund exhibitiing an improvement in the one or more ADMET/PK properties over the therapeutic chemical compound, while substantially preserving a therapeutic effectiveness exhibited by the therapeutic chemical compound.

The therapeutic chemical compound may be a drug approved for sale by the U.S. Food and Drug Administration. It may have a demonstrated side effect attributable to one or more of its ADMET/PK properties. The model may preduct a metabolic property of the terapeautic chemical compound, for example, in the form of separate lability values for individual reactive sites on the therapeutic chemical compound. The modification may involve one or more of the reactive sites to thereby speed up or slow down the rate at which the therapeutic chemical compound is metabolized. The model may predict binking affinity of the therapeutic compoound to a P450 isozyme. The model may predict lability values for individual reactive sites on the therapeutic expression developed from a regression analysis performed on data associating reactivity with structural features.

The method may involve identifying one or more features of the therapeutic chemical compound that react most rapidly during metabolism, and comprises specifying one or more modifications of the one or more structural features. The model may prodict how rapidly chemical compounds perneate cell layers. Some or all of the operations may be performed as software executing one or more processors. The therapeutic chemical compound may be received over the Internet. The therapeutic chemicla may be selected from the group consisting of allopurinol, aniodarone, chloramphenicol, cipofloxacin, clarithomycin, diltiazem, dirithromycin, disulfiram, enoxacin, erythromycin, fluconazole, fluoxetine, fluvoxamine, isoniazid, itraconazole, ketoconazole, metranidazole, miconazole, MAOIs, nefazodone, omeprazole, trimehtoprim/sulfamethoxazole, troleandomycin, verapamil, propoxyphene and quinidine.

A modification may reduce the therapeutic chemical compound's inhibitory effect on a CYP450 enxyme, example compounds being acetaminophen, amobarbital, aprobarbital, butabarbital, butalbital, carbamizepine, efavirenz, ethotoin, fosphenytoin, mephenytoin, mephobarbital, phenobarbital, phenytoin, primidone, rifabutin, rifampin, rifapentine and secobarbital. A modification may reduce the therapeutic chemical compound's toxicity example compounds being troglitazone, bromfenac, atrovastatin, zenarestat, trovafloxacin, cisapride, adefovir/dipivoxil, tolcapone, naproxen, ibuprofen and phentermine. The method may include analyzing the structure of the therapeutic chemical compound with multiple different models sequentially or in parallel, each model predicting one or more ADMET/PK propertics of chemical compounds.

In predicting absorption, the absorption property may have at least one component selected from the following: solubility, passive permcation of cell membranes, passive intestinal absorption, active transport across cell membranes. In predicting metabolism, the metabolism property may have at least one component selected from the following: binding affinity to metabolizing enzyme, rate of metabolism and regiolability. In predicting excretion, the excretion property may have at least one component selected from the following: hepatic excretion adn renal excreation. In prodicition toxicity, the toxicity property may have at least one component selctied from the following: bioativation of toxic metiabolic intermediates, and structural features in the parent compound that are correlated with toxicity. In predicting distribution, the distribution property may have at least on component selected from the following: blood-brain-barrier penatration, human serum albumin binding, protein binding, receptor binding and transport across cell membranes. The methods may also include simultaneously displaying the analyzed ADMET/PK properties of a library of compounds.

Another aspect of the invention pertains to an apparatus for analyzing therapeutic chemical compounds in order ot redesign the therapeutic chemical compounds, the apparatus including an iterface for receiving information, including the identity of chemical compounds, from at least one external source, a memory device for storing, at least temporarily, chemical structural information of a therapeutic chemical compound that is a drug candidate, drug or withdrawn drug and instructions for analyzing one or more ADMET/PK properties of the therapeutic chemical compound, and one or more processors desgned or configured to identify one or more structural features of the therapeutic chemical compound that cause the therapeutic chemical compound to possess an ADMET/PK property that can be improved, and specify a modification for the one or more structural features to produce a modified chemical compound exhibiting an improvement in the one or more ADMET/PK properties over the therapeutic chemical compound, while substantially preserving a therapeutic effectiveness exhibited by the therapeutic chemical compound.

The one or more processors may be configured to perform some of these operations executing said instructions stored in memory and may beconfigured with a software application or an integrated suite of software tools for analyzing the therapeutic shemical compounds. The interface may allow connection to the Internet so that the therapeutic chemical compound can be identified by receiving the compound's identity over the Internet. The apparatus may also include a software program for providing a structural representation of the therapeutic chemical compoound from the identity of the therapeutic chemical compoound. compound classes that eventually lead to a single marketable drug or possibly a few such drugs.

Those thousands of candidate compounds are screened against biochemical targets to assess whether they have the pharmacological properties that the researchers are seeking. This screening process leads to a much smaller number of “hits” (perhaps 500 or 1000) which display some amount of the desired properties, which are narrowed to even fewer “leads” (perhaps 50 or 100) which are more efficacious. At this point, typically, the lead compounds are assayed for their ADMET/PK (absorption, distribution, metabolism, elimination, toxicity; i.e., pharmacokinetic) properties. They are tested using biochemical assays such as Human Serum Albumin binding, chemical assays such as pK_{A} and solubility testing, and in vitro biological assays such as metabolism by endoplasmic reticulum fractions of human liver, in order to estimate their actual in vivo ADMET/PK properties. Most of these compounds are discarded because of unacceptable ADMET/PK properties.

Once a lead compound has been identified, structural or functional analogs of the lead compound can be generated. These analogs can then be screened to identify additional lead compounds, and to obtain additional structure-activity relationship (SAR) data that may be useful in the design of still-further optimized leads. The iterative process of lead compound selection, analog generation, and screening of the analogs to identify further lead compounds can be repeated as many times as desired, until an optimized lead compound is obtained.

Selection of analogs for lead generation or optimization involves balancing of considerations such as potency, selectivity, ADMET characteristics, and the like. Traditionally, medicinal chemists have selected compounds according to their knowledge of structure-activity relationships, and, as such, this process has often been rather subjective. As described in more detail herein, some of these considerations can be automated and performed by, e.g., software capable of calculating metabolic labilities for candidate drugs. Thus, computational techniques for predicting potency, selectivity, solubility, or other parameters can be employed (in silico) for selecting the most promising compounds for synthesis.

Even optimized leads that have passed these tests and are submitted for FDA clinical trials as investigational new drugs (INDs) will sometimes show undesirable ADMET/PK properties when actually tested in animals and humans. Abandonment or redesign of optimized leads at this stage is extremely costly, since FDA trials require formulation, manufacturing and extensive testing of the compounds.

The one or more processors may be designed or configured to predict a metabolic property of the therapeutic chemical compound. They may be designed or configured to predict the metabolic property in the form of separate lability values for individual reactive sites on the therapeutic chemical compound. They may be designed or configured to specify a modification for the one or more structural features by modifying one or more of the reactive sites to thereby speed up or slow down the rate at which the therapeutic chemical compound is metabolized. They may be designed or configured to predict lability values for individual reactive sites on the therapeutic chemical compound by employing at least one of a quantum mechanical model or an expression developed from a regression analysis performed on data associating reactivity with structural features. They may be designed or configured to predict how rapidly chemical compounds are absorbed into the blood stream.

Another aspect of the invention pertains to methods for analyzing a therapeutic chemical compound to redesign the therapeutic chemical compound, including identifying a therapeutic chemical compound that is a drug candidate, drug or withdrawn drug, utilizing a software application or integrated suite of software applications to analyze the therapeutic chemical compound, wherein the software application or integrated suite of software applications can analyze at least two of the following properties of the therapeutic chemical compound: absorption, metabolism, distribution and toxicity, and specifying a modification to the therapeutic compound in order to modify at least one of the following properties of the therapeutic compound: absorption, metabolism, distribution, excretion, and toxicity. The methods may include additional aspects as described in the above aspects of the invention.

Another aspect of the invention pertains to an apparatus for analyzing therapeutic chemical compounds to redesign the therapeutic chemical compounds, the apparatus including an interface for receiving information, including the identity of chemical compounds, from at least one external source, a memory device for storing, at least temporarily, chemical structural information of a therapeutic chemical compound that is a drug candidate, drug or withdrawn drug and instructions for analyzing at least two of the following properties of the therapeutic chemical compound: absorption, metabolism, distribution, toxicity and solubility, and one or more processors designed or configured to analyze the therapeutic chemical compound stored in memory by evaluating at least two of the following properties of the therapeutic chemical compound: absorption, metabolism, distribution, toxicity and solubility, and specify a modification to the therapeutic compound in order to modify at least one of the following properties of the therapeutic compound: absorption, metabolism, distribution and toxicity. The apparatus may include additional aspects as described in the above aspects of the invention.

Another aspect of the invention pertains to computer program products including a machine-readable medium on which is stored program instructions for implementing any of the methods described above. Any of the methods of this invention may be represented as program instructions that can be provided on such computer readable media.

These and other features of the present invention will be described in more detail below in the detailed description of the invention and in conjunction with the following figures.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A is a high-level flowchart describing one method for redesigning or “rescuing” a therapeutic compound.

FIG. 1B is a high-level flowchart describing a second method for rescuing a therapeutic compound.

FIG. 2 is a schematic illustration of a substrate molecule (drug) with several reactive sites.

FIG. 3 is a block diagram that illustrates a preferred embodiment of the integrated suite of software applications to redesign a therapeutic compound.

FIG. 4 is a block diagram that illustrates a preferred embodiment of the process for modeling a protein-drug interaction, e.g., active transport docking.

FIG. 5 is a schematic illustration of the mammalian cyctochrome p450 catalytic cycle, including the non-metabolic decoupling reactions.

FIGS. 6A and 6B together make up a flowchart for analyzing the metabolic properties of a therapeutic compound as part of the redesign process.

FIG. 7 is a schematic illustration of a sample generic drug molecule and accompanying regiolability table, Metabolic Landscape™.

FIG. 8 is a table listing several benzodiazapene compounds, their Metabolic Landscapes™ and labile sites and determined by the current invention, and their observed bioavailabilities and half lives.

FIG. 9A displays potential rescue analogs to fluoxetine and their predicted CYP2D6 binding affinities.

FIG. 9B is a table showing the go/no-go CYP2D6 binding affinity values.

FIG. 10A displays potential rescue analogs to tegaserod and their predicted log BBB values.

FIG. 10B is an illustration showing the structure of imatinib, a compound identified for rescue.

FIGS. 11A and 11B illustrate a computer system suitable for implementing embodiments of the present invention.

FIG. 12 is a block diagram of an Internet based system for “rescuing” therapeutic compounds in accordance with an embodiment of the present invention.

parent compound may be too toxic but otherwise have fine ADMET/PK properties. If the chosen compound is significantly less toxic but slightly poorer in absorption, the methods of this invention should not disregard the new compound simply because it has poorer absorption.

Preferably, the invention selects only those compounds that are highly likely to preserve the therapeutic effectiveness of the parent compound. There is not always a foolproof technique for accomplishing this. Generally, the activity of a compound is associated with a particular combination of functional sites that might interact with a particular receptor molecule. Various QSAR (Quantitative Structure Activity Relationship) tools may be employed to filter proposed compounds based upon their likely activity. Other tools known to those of skill in the art may be employed to filter likely inefficacious compounds, including empirical assays, preferably those with high-throughput, that are well-known to those of skill in the art. There are also software programs well-known to those of skill in the art that can generate candidate compounds that are structurally related to the parent, for example, MODDE and SIMCA available from Umetrics, Inc., of Umea, Sweden.

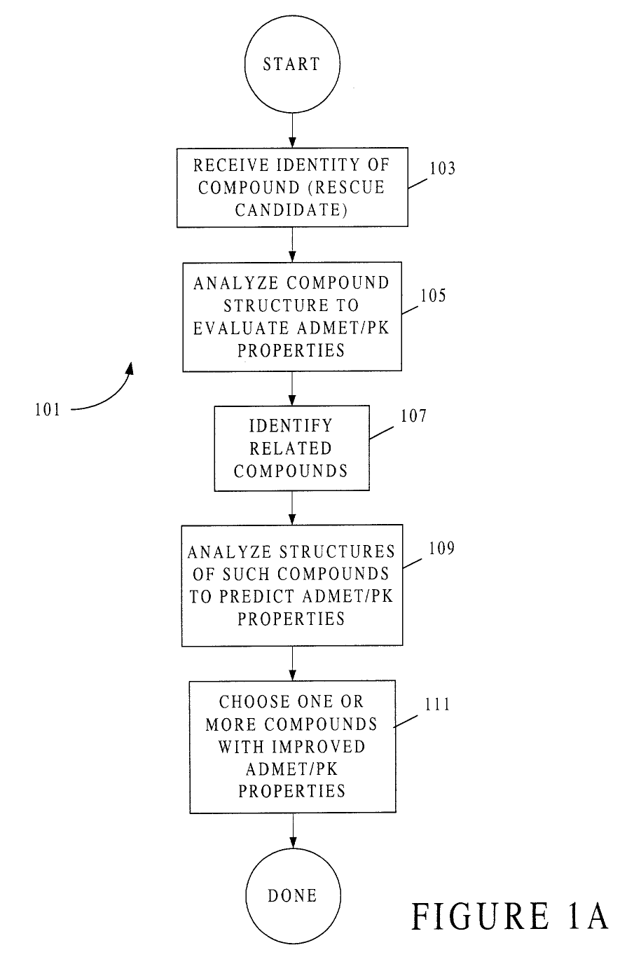

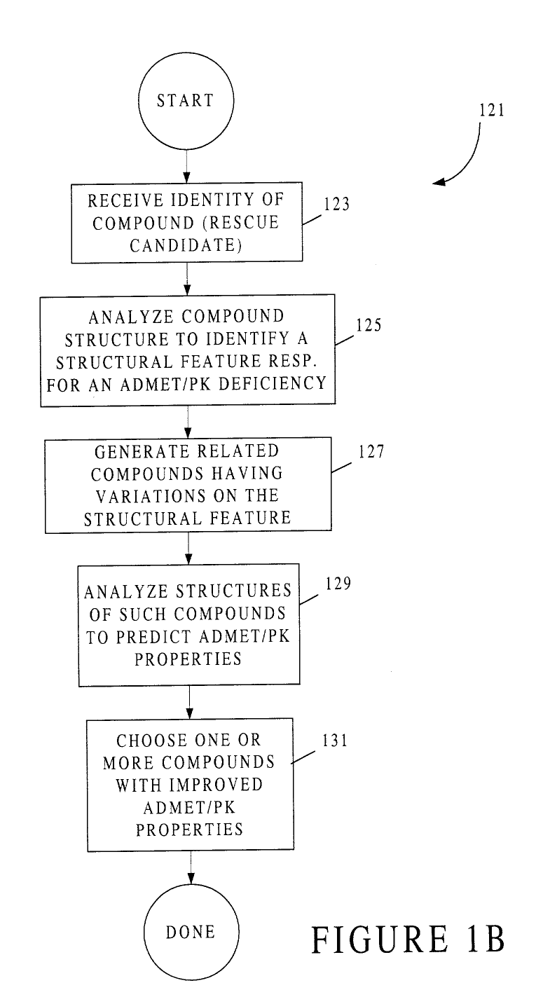

FIG. 1B is a separate flow chart depicting a slightly different methodology of this invention, again presented from a very high level. Any number of the described method operations may be performed with a computational device. And one or more of such operations may be dispensed with as is appropriate for the particular project at hand and available system resources.

The depicted methodology, 121, begins as before with receipt of the identity of a parent compound for rescue. See 123. Next, the computational system analyzes the compound to identify one or more structural features that may be responsible for an ADMET/PK deficiency. See 125. Rather than predicting an overall value for the compound's ADMET/PK property (as may be done at 105 in process 101), the system specifically flags a problem site on the compound. This may be a highly labile site, for example, that causes the compound to metabolize too quickly.

After the problem site has been identified, the methodology identifies or generates one or more related compounds having variations on the chemical structure present at the problem site. See 127. For example, if the site possesses a vinyl group, the computational system may generate analogs in which the vinyl group is replaced with conjugated groups, ethers, etc. After the full complement of candidates has been generated at 127, the methodology analyzes the structure of each such compound to predict one or more ADMET/PK properties. See 129. This may be accomplished in the manner described for operations 105 and 109 of methodology 101. Finally, one or more of the candidates provided at 127 is chosen at 131. The selection criteria may be those employed for operation 111 of methodology 101.

Central to this invention is selection of therapeutic compounds for rescue. Information pertaining to selection of such compounds will be detailed below. For now, understand that such compounds should have exhibited a therapeutic effectiveness through clinical trials or longstanding use, for example. Many of the rescue candidates will be legally marketed and approved by the US FDA or other governmental body. The candidate compounds should also have an ADMET/PK deficiency susceptible of improvement through structural modification. Particular ADMET/PK deficiencies for a given therapeutic compound may be recognized in the field or may be elucidated using tools of this invention.

Selection or identification of potential rescue compounds may be accomplished via a systematic search and analysis. For example, human or a software agent may explore various databases of compounds to identify potential therapeutic compounds for rescue. The search may focus on a particular class of compounds. The class may be defined structurally or functionally. A few examples of functional classes include CYP 450 inhibitors, CYP 450 inducers, and toxic compounds. A few examples of structural classes include statin compounds and benzodiazapines. Potential rescue compounds may also be chosen on the basis of their therapeutic potential and even market performance, and useful classes of drugs may even be defined by such considerations.

In the methodologies of this invention, the identity of the parent chemical compound may be received alone or in conjunction with varying levels of structural information. In the simplest case, the identity is provided merely as a numerical or textual identifier (possibly the IUPAC name). In a more sophisticated example, the chemical identity is provided with some additional information such as the two-dimensional or three-dimensional structures of the compound. Details on atomic coordinates, bond energies, etc. may be supplied. Some of this information can be employed to generate multiple conformations of a given compound.

When very limited structural information is received with a compound's identity, the systems of this invention typically must supply additional structural information necessary to analyze the compound and suggest structural modifications. Thus, the computational systems of this invention may include logic for generating full structural representations of chemical compounds. The logic may be developed from scratch or may be an off the shelf product. Preferably, the logic can handle structures received as an organic chemistry string of atoms, a two-dimensional structure, a IUPAC standard name, a 3D coordinate map, or as any other commonly used representation.

Examples of available systems for generating a 3D coordinate map of the molecule include geometry programs such as Corina or Concord. The 3D structure generator Corina is available from Molecular Simulations, Inc., of San Diego, Calif. and Molecular Networks GmbH of Erlange, Germany. Concord is available from Tripos, Inc. of St. Louis, Mo. Corina uses straightforward rules about molecular bond and functional group conformation to generate an approximate geometry 3D structure, which is optimized to a local energy minimum. For instance, if an amine group is encountered, then it will be placed in a planar conformation, as that group normally exists. Concord applies a similar method, but also uses a limited set of molecular mechanical rules involving branch angles, strain and torsion, to achieve its 3D structure.

Depending upon the needs of the particular ADMET/PK model, an approximate 3D geometry structure may be optimized with a more sophisticated modeling tool, such as AM1. AM1 is a semi-empirical quantum-chemical modeling program that optimizes the given 3D structure to that local energy minimum. It calculates electron density distributions from approximate molecular orbitals. It also calculates an enthalpy value for the molecule. AM1 is available as part of the public-domain software package MOPAC, which is available from the Quantum Chemistry Program Exchange, Department of Chemistry, Indiana University, Bloomington, Ind. The MOPAC-2000 version of MOPAC can be obtained from Schrödinger, Inc., of Portland, Oreg.

As for generating compounds that are related to the compound being redesigned, known compounds of a similar structure and similar therapeutic effect can be taken from any source, such as a biochemical database. Alternatively, compound structures can be generated de novo from the original structure of the chosen therapeutic compound. Various software tools can be used to perform this task, for example, MODDE and SIMCA available from Umetrics, Inc., of Umea, Sweden. Both of these alternatives also can be employed together.

As for selecting promising candidates previously screened to meet particular ADMET/PK requirements, the therapeutic effectiveness of those candidates may be assessed. The activity of a compound is associated with a particular combination of functional sites that might interact with a particular receptor molecule. Various QSAR (Quantitative Structure Activity Relationship) tools may be employed to filter proposed compounds based upon their likely activity. Other tools known to those of skill in the

Models for Analyzing ADMET/PK Properties

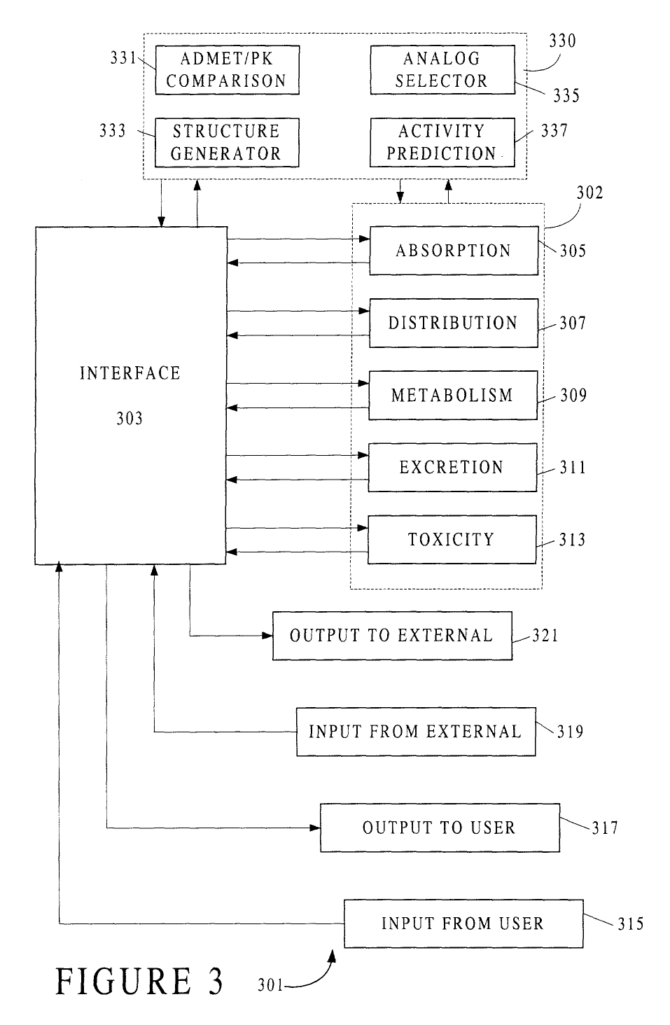

Various ADMET/PK models may be employed with this invention. The term “model” may refer to any method or system that can predict an ADMET/PK property (or related property) based on chemical structural information. As suggested above in the discussion of FIG. 3, computational systems of this invention may be provided with one or more of such models. Individual models may predict individual ADMET/PK properties. Or they may predict combinations of ADMET/PK properties. The ADMET/PK properties that may be analyzed include absorption (including but not limited to solubility, passive permeation of cell membranes, passive human and animal intestinal absorption, active transport across human and animal cell membranes), distribution (including but not limited to blood-brain-barrier penetration, human serum albumin binding, protein binding, receptor binding, drug transport across cell membranes), metabolism (including but not limited to compound binding affinity to metabolizing enzyme, rate of metabolism, regiolability, regiospecificity), excretion (including but not limited to hepatic and renal excretion), and toxicity (including but not limited to bioactivation of toxic metabolic intermediates, identification of structural features in the parent compound that are correlated with toxicity).

Each of the ADMET/PK properties can be analyzed using the current invention. Each of these properties is typically be analyzed using a computational model based on software, for instance, the absorption software application. Each software application will typically composed of sub-modules running in parallel, wherein each sub-module analyze an aspect of that property, for instance, the passive permeation of cell membranes aspect of absorption. All the software application will typically be incorporated into an integrated suite of software applications.

Regardless of which properties an individual model predicts, it should generally make that prediction based upon the chemical structure of the compound under consideration. In some cases, the model will rely on a full two-dimensional or three-dimensional representation of the compound. In other cases, the model will require only a partial two-dimensional or three-dimensional representation. This may be true when only a portion of the compound is available for redesign. Other types of models will require only “structural descriptors” of the input compounds. These may describe a molecular fragment, a molecular site, or geometric parameter of the molecule.

Structural descriptors have been found to be particularly useful in generating some models, particularly regression-based models. They represent ‘important’ structural features of molecules. These features are likely to influence the reactivity (or other ADMET/PK property) of a particular site on the compound. Examples of site-specific structural properties captured in descriptors include (a) the classification of a site according to atom type and electronic hybridization, (b) the influence of neighboring atoms and groups, (c) the geometric constraints on the site resulting from participation in a ring, size of the ring, and/or flexibility of the ring, and (d) the partial and/or formal charge on the atom at the site (or elsewhere on the molecule). For further discussion on how certain descriptors that are appropriate to the current invention can be used, see U.S. patent application Ser. No. 09/811,283 (Atty Docket No.: CAMIP005).

The influence of neighboring atoms may be captured with descriptors that characterize electron withdrawing properties of neighbors, participation in a conjugation system, participation in a ring system, etc. The geometric state of a site may be captured using descriptors that specify steric factors hindering or facilitating accessibility to a particular site. These factors may result from neighboring structures on the molecule or the relative geometric positioning of a particular site (e.g., at the end of a major axis on an ellipsoid shaped compound). The partial charge on an atom reflects the degree to which the atom donates its electrons to (or receives electrons from) neighboring atoms. More details associated with structural descriptors, particularly as used for CYP metabolism models, are provided in U.S. patent application Ser. No. 09/811,283 (Atty Docket No.: CAMIP005), previously incorporated by reference.

For ADMET/PK properties typically affected by a group of sites (e.g., adsorption), groups of structural descriptors may be employed. In another specific embodiment, the descriptors identify relevant fragments of a molecule. A system generating such fragments takes, as an input, a molecular structure and applies a set of fragmentation rules. Generally, such rules fragment a molecular structure in a manner that preserves in the resulting fragments the important descriptor information identified above. The fragment sizes are chosen to be relevant to the particular ADMET/PK property under consideration. In some cases, the only meaningful way to consider the ADMET/PK property of a compound is by considering the entire structure of the compound, via its detailed three-dimensional structure, for example.

The mathematical or algorithmic underpinnings of models can take various forms. This variation is independent of the type of structural representation or descriptor used as an input to the ADMET/PK models. Examples of model formats include mathematical expressions (e.g., linear or non-linear single or multi-order equations), neural networks, trees or graphs, etc.

In one embodiment, quantum chemical techniques model the electronic configurations and energies associated with atomic orientations. The essence of a quantum chemical method involves calculating the electronic structure of a given atomic configuration. The electronic configuration of a molecule is obtained by combining atomic orbitals to form molecular orbitals. The equations for the electronic waveforms have been around since the beginning of the twentieth century, but they are not amenable to solution. Therefore different approximations such as semi-empirical methods (using experimental data) and ab initio methods (using a basis set of Gaussian functions to approximate atomic orbitals) are used in the solution to these equations. Approximate geometries are optimized to stable geometries by minimizing the energy with respect to the atomic coordinates. Various ADMET/PK properties can be modeled using quantum chemical techniques. In one example, reactions are modeled by transforming the reactant geometry to the product geometry and minimizing all but one degree of freedom. Additional details of quantum chemical modeling of metabolism can be found in U.S. patent applications Ser. Nos. 09/368,511 (Atty Docket No.: CAMIP001), and 09/613,875 (Atty Docket No.: CAMIP002).

As mentioned, the model can take the form of a specific expression for site reactivity. Often such expressions will be comprised of a sum of terms, each representing a different structural descriptor. A separate coefficient may be provided for each such term. Often such expressions derive from a statistical technique such as a multivariate regression analysis.

Generation of a statistical model (or a neural network) requires the use of a training set. This is a sample group of compounds having known or reliably predicted ADMET/PK properties. The training set should have a wide range of chemical structures and ADMET/PK properties.

In a preferred embodiment of the invention, a set of fragments or geometry descriptors are determined for a training set of molecules. The ADMET/PK properties of interest for the molecules are predetermined from an external method, such as actual experimental measurements or more computationally intensive estimates, e.g., quantum chemical modeling. The sites of the molecules are described in terms of the descriptors chosen, and a linear regression analysis or other fitting is done to create a simple relationship between descriptor values and ADMET/PK property. In a specific embodiment, the relationship is a linear equation with coefficients that match the ADMET/PK data of the training set with a least squares fit. The linear equation is then applied to subsequent molecules to model and predict their ADMET/PK properties. The reactive sites are typically binned into three categories: labile, moderately labile and stabile. For further discussion on the relative rates curve and the meaning of these lability categories, see U.S. patent application Ser. No. 09/613,875 (Atty Docket No.: CAMIP002). While the methods discussed above and discussed in the Patent Application are the most important to determining the absolute rate of metabolism of a drug, some stoichiometry-based techniques can be useful for determining the absolute rate of metabolism, as well as the absolute rate of the ADMET/PK properties in general.

The next operation is an optional steric and orientation factor correction operation. See 623. As stated earlier, the CYP enzymes, particularly 3A4, are not sterically specific in the way that other enzymes are. However, in certain cases, a reactive site may be deeply buried within the substrate molecule, or the molecule may have a strongly preferred amphoteric orientation, so that the relative rate of the reactive site in metabolism is hindered or accelerated. In such cases, the user may wish to incorporate steric or orientation correction factors. See 625. For further discussion of steric and amphoteric correction factors, see U.S. Provisional Patent Application Patent Application No. 60/217,227 and U.S. patent application Ser. No. 09/902,470, both previously incorporated by reference.

In some embodiments, it will be desirable to have separate metabolism models for individual CYP enzymes. This is because some compounds are metabolized exclusively, or nearly exclusively, by a single CYP enzyme. In such cases, the specificity afforded by a model of the single CYP enzyme can provide valuable insight into a compound's overall metabolism and potential interaction with other compounds. Such models may include differing accessibility criteria, for example, as described in U.S. patent application Ser. No. 09/902,470.

For various reasons, it is desirable to have compounds that are metabolized by multiple CYP enzymes. To this end, redesign efforts of this invention may modify the structure of a base compound that is metabolized primarily by a single enzyme so that it can now be metabolized by multiple enzymes. Such redesign efforts may make use of multiple metabolism models, each directed to a different CYP enzyme as mentioned.

In a preferred embodiment, descriptor-based models for binding affinities are also incorporated into the model. In some embodiments, algorithms calculate up to 600 different 2-dimensional and 3-dimensional descriptors. These descriptors represent individual or composite molecular properties and were used to relate structure to experimentally determined Kᵢ values using molecular modeling techniques and multivariant statistics with software packages, for example, MODDE and SIMCA available from Umetrics, Inc., of Umea, Sweden.

The above procedure is used to predict the metabolism rate and/or profile for the original rescue candidate as well as the proposed substitute compounds. Any of the proposed substitutes having superior metabolic properties may represent a viable alternative to the original rescue candidate. It is worth noting that the core process for determining the relative rates is carried out without reference to the CYP enzymes or any other specific enzymes. As long as the enzymes being studied carry out metabolism by similar mechanisms, with the same transition states of the substrate being created, the data from one analysis of relative rates can usefully be applied to other enzymes.

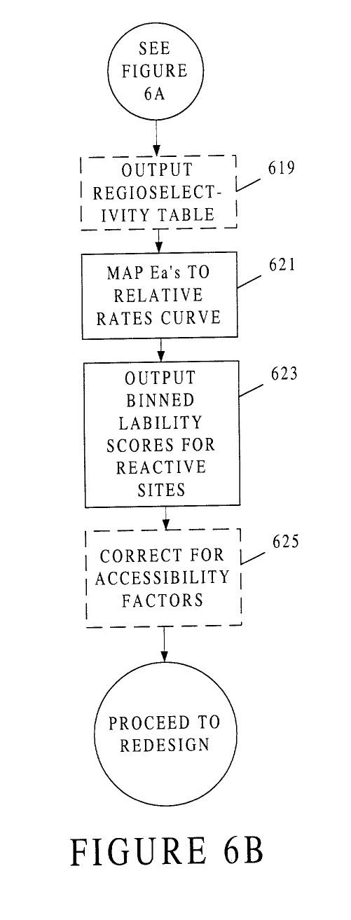

A form of output from the metabolic modeling described above is a regiolability model, which typically includes a user-friendly output such as the Metabolic Landscape™ output, which is a trademark of Camitro Corporation of Menlo Park, Calif. See FIG. 7, 700. Each bar in the graph, for example 703, corresponds to a reactive site, for example 705, on the sample generic molecule 707. Each bar 703 is typically labeled with its relevant carbon atom. In FIG. 7, there are 18 reactive sites represented in the graph, ordered from most labile to most stable. The reactive sites are typically binned into four categories; labile, moderately labile, moderately stable and stable. The reactive sites in this example are normalized, with the value for the most labile site being 100%. For further discussion of relative labilities of reactive sites and typical activation energy values that correspond to these bin categories, see U.S. patent applications Ser. No. 09/613,875 (Atty Docket No.: CAMIP002).

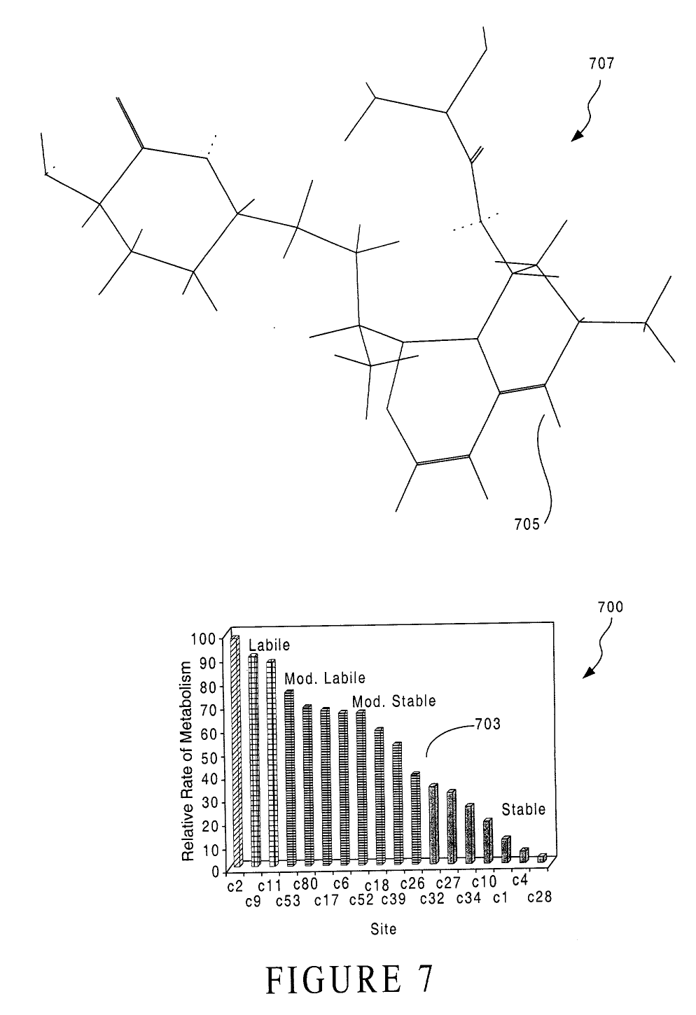

FIG. 8 is a table 800 of several benzodiazepine compounds, including adinazolam 801 and midazolam 803. Adinazolam 801 and midazolam 803 were determined to have two and one labile sites, respectively, as determined by the current invention and listed in column 805. The number of labile sites is also clearly visible in the fingerprint versions of the Metabolic Landscape™ curves listed in column 811. FIG. 8 also serves to illustrate the robustness of the current invention, in that the two compounds adinazolam 803 and midazolam 805 that were determined to have labile sites were experimentally-observed to have low bioavailability and short half lives, as one would expect. See 807 and 809. In the absence of other factors, compounds with more labile sites are expected to be shorter-lived in vivo than compounds with fewer labile sites. Generally, bioavailability refers to the percentage of an administered compound that is available in the blood stream for therapeutic effect. Thus, it is a measure of a compound's ability to (1) pass through the intestinal lining and (2) remain free in the blood stream, unbound to blood components such as human serum albumin.

Absorption, Distribution and Excretion Analysis and Redesign

In a preferred embodiment, absorption, distribution and excretion analysis is carried out using a single software application, since there are common aspects of the analytic models required for each of these properties. However, other embodiments call for these to each have their own software application.

Absorption

With regard to intestinal absorption, the drug must cross the epithelial cells that line the gastrointestinal tract, and for distribution, they must cross the endothelial cells that line the blood capillaries that perfuse the target organs. Such traversing can occur via the paracellular (in between cells) or the transcellular (through cells) pathway. Since only small hydrophilic molecules (MW less than 350) can use the paracellular route, the majority of compounds cross cell membrane barriers via transcellular routes. Transcellular routes include passive diffusion and carrier-mediated (or active) transport.

Most drugs and drug candidates will fall into the higher MW category, so that the paracellular sub-module will not be run or will return a null value. The transcellular sub-module models transport-independent (passive) processes and involve the use of standard descriptors well-known in the art that represent the key transitions in the permeability process. Modeling of and transport-dependent processes focuses on modeling the transport-protein interactions.

At the intestinal epithelium, compounds are primarily absorbed via passive diffusion across the vast surface area of the densely packed microvilli at the apical brush border membrane, and via transport mediated by selective transporter proteins. However, this import is counter-acted by efflux mechanisms that transport compounds out of the cell back into the intestinal lumen. One such efflux mechanism is mediated by the transporter protein P-glycoprotein (P-gp), also called the multidrug transporter (MDR1). P-gp is the most important transported protein in the transport-dependent model of the transcellular sub-module. P-gp is a member of a highly conserved group of energy-dependent ATP-binding-cassette (ABC) transporters found in a wide range of cell types. database of drug adverse events reported by health professionals and others on marketed drugs. The database is currently growing at a rate of approximately 300,000 reports annually. It contains compound structures with associated adverse events information encoded with either Event Codes in COSTART (Coding Symbols for Thesaurus of Adverse Reaction Terms) (used up until 1997) or with Preferred Term Event Codes in MedDRA (Medical Dictionary for RegulatoryActivities) (used after 1997). Both COSTART and MedDRA are hierarchical coding systems, with multiple levels of term grouping, from (in the case of MedDRA) “lower-level terms” to “primary terms” to “higher-level terms” to “higher-level-group terms” to “system-organ-classes.”

The AERS database is the accumulation of numerous spontaneous adverse event reports, each of which enumerates one or more suspected drugs and one or more suspected adverse reactions. This data is easily transformed into a large two-way table of counts; i.e. the number of times a given drug and a given event are mentioned together in the same report, for all distinct pairs of drugs and events. Numerical strength-of-association scores (“signal scores”) can be assigned based only on this raw count data. This generates a Bayesian shrinkage estimate for the ratio of the “observed” to the “expected” count for each combination of drug and event (the “expected” count is the count arising from the separate marginal distributions of drug and event counts along with an assumption of independence). One example of software that generates data under such an approach, known as the DuMouchel algorithm, is available from Lincoln Technologies of Weston Newton, Mass.

In a preferred embodiment, each MedDRA adverse event codes is categorized by organ-specific type of toxicity (hepatic, renal, cardiac, neurological, etc.), based on the built-in hierarchical organization of the MedDRA terminology, in which Primary Terms are linked to Higher Level Terms that are in turn linked to Higher Level Group Terms that are finally linked to 26 top-level System Organ Classes. For each set of MedDRA terms making up a “toxicity group”, a weighted average of the corresponding “signal scores” is used to produce a corresponding “toxicity score.” The resulting database contains a set of organ-specific toxicity scores for each drug appearing in the adverse events database.

A database such as of the MOE (Molecular Operating Environment) software package available from Chemical Computing Group Inc. of Montreal, Canada can be used to store the substructure elements of parent compounds with reactive intermediates as well as their associated toxicity data. The toxicity software module will thus work on the basis of a substructure searching functionality and the substructure-toxicity database and will systematically screen query compounds for their reactive intermediate potential. Upon identification of a relevant substructure, the model accesses the predictive metabolism model and determines the relative likelihood for metabolism to occur at that particular site and to give rise to the reactive intermediate responsible for toxicity.

This process is automated, in a preferred embodiment of the invention, with a computational engine that flags compounds with potentially reactive functionalities that are predicted to be significantly metabolized by a P450 major isoform. With toxicity scores computed for each of several organ systems for each of approximately 1,000 compounds, statistical modeling techniques are used to produce QSAR models that predict each of the types of toxicity from chemical structure. In order to develop the QSAR model, “recursive partitioning” of molecular features is used. Recursive partitioning involves linear and non-linear relationships to describe combinations of substructures and other molecular features that are responsible for activity. Recursive partitioning methods result in a dendrogram, in which structural characteristics responsible for activity (or inactivity) are associated with branches of the dendrogram. The splits to active and inactive branches or terminal nodes give information about the structural requirements for activity. This QSAR information can then be used to identify which metabolites and intermediates produced from the drug (as predicted by the metabolism model) are in fact toxic.

An example of toxicity caused by reactive intermediate formation is the drug troglitazone. Rezulin is one of the highest profile cases of a drug failure due to ADMET/PK properties. The method of the current invention is successful at predicting and modeling Rezulin's toxicity. Troglitazone causes time and NADPH-dependent inhibition of CYP 3A4, indicating that a reactive intermediate is formed by CYP 3A4 metabolism. At the 2001 ISSX meeting, Tom Baillie (VP, Preclinical Research at Merck) reported that the hepatotoxicity of troglitazone was mediated by the initial CYP 3A4-mediated sulfur oxidation reaction. The current invention accurately predicts the susceptibility of troglitazone to metabolism at the site on the molecule that leads to hepatotoxicity from the sulfur oxidation intermediate. The current invention predicts that this intermediate accounts for 60% of metabolism, and that 40% of metabolites are distributed among a subset of other intermediates. From a drug rescue perspective, the ability to model this presumed toxic pathway provides a method by which analogs that are devoid of this deficiency can be designed and tested.

Identification of Classes of Compounds for Redesign and Choosing Redesign Compounds

As mentioned above, this invention pertains to rescue of compounds for which a therapeutic benefit has already been discovered, but for which an ADMET/PK deficiency exists. Usually that deficiency will be known and it may have already caused the compound to be withdrawn from public use or sidetracked on its way to approval. Hence many of the compounds used with this invention will fall into one of the following classes: ethical pharmaceutical drugs, withdrawn drugs, and lead compounds.

To identify candidates for rescue, one may systematically consider various categories of therapeutic compound. Some structural or functional categories are likely provide more rescue candidates than others. The class of interest may contain only compounds known to have a particular functional effect on a particular ADMET/PK property. One such class includes compounds that “induce” metabolism by certain metabolic enzymes. Another class includes compounds that “inhibit” metabolism by certain metabolic enzymes. Yet another class includes compounds that provide a particular toxic metabolite. Still other compounds are selected based on market and therapeutic value.

Aside from locating rescue candidates by identifying compounds known to have a particular ADMET/PK related property, one can locate candidates by a particular structural or functional classification not directly related to ADMET/PK properties. Structural classifications may be based on a common chemical “scaffold” (e.g., statin compounds and benzodiazepines) or a chemical binding site (e.g., MAO inhibitors and histamine H2 receptor antagonists). Some of these classes will have many members that could profit from ADMET/PK driven redesign. For example, many statin compounds have low oral bioavailability and short half-lives due to rapid metabolism. The computational methods of this invention can identify members of the class having improved ADMET/PK properties. In some cases, however, the methods will show that all members of a class are susceptible to a particular ADMET/PK problem. For example, all statins might have a labile site that is central to the pharmacological activity of the class. In that case, the invention will suggest to the chemist that improvements in phrarmacokinetics may have to be obtained from an avenue other than imparting metabolic stability.

One class of compounds that can be considered for drug rescue includes those therapeutic compounds known to (or determined to) suffer drug-drug interactions. Among the examples of marketed drugs in this class are the following: allopurinol, accumulates in the body to toxic levels and causes harm. Examples of such inhibitors include the following compounds on the market: amiodarone, chloramphenicol, cimetidine, diltiazem, fluoxetine, fluvoxamine, indinavir, itraconazole, ketoconazolemibefradil, paroxetine, propafenone, quinidine, ritonavir, sertraline and troleandomycin.

Another class of compounds that can be considered for drug rescue includes therapeutic compounds that induce activity of P450 enzymes. These compounds also interfere with the metabolism of a co-administered drug, but they do it by inducing the activity or expression level of the respective P450 enzyme, thus increasing the metabolism of the co-administered drug and leading to its rapid inactivation and removal. Examples of such inducers include the following compounds on the market: acetaminophen, amobarbital, aprobarbital, butabarbital, butalbital, carbamazepine, efavirenz, ethotoin, fosphenytoin, mephenytoin, mephobarbital, phenobarbital, phenytoin, primidone, rifabutin, rifampin, rifapentine and secobarbital.

Another class of compounds that can be considered for drug rescue includes therapeutic compounds that are believed to be intrinsically toxic and/or to produce a toxic metabolite. Examples of such toxic compounds include the following compounds on the market: troglitazone, bromfenac, atorvastatin, zenarestat, trovafloxacin, cisapride, adefovir dipivoxil, tolcapone, naproxen, ibuprofen and phentermine.

In this invention, the redesign candidates can be provided in two general ways. First, a moiety or moieties identified as responsible for an inferior ADMET/PK property can be modified to suggest a candidate compound that should have improved ADMET/PK properties. Second, a series of related analogs can be produced and “screened” for both therapeutic activity and ADMET/PK suitability.

Analogs of a compound for redesign may share a core structure in common with the base compound (e.g., if the compound is a benzofuran compound, the analogs may also be benzofurans, differing from the lead compound in any substituents appended to the benzofuran moiety). Alternatively, the analogs can differ from the base compound in some way, preferably in such fashion that the analogs retain some desirable characteristics of that compound. For example, if the base compound is a benzofuran, the analogs may be benzothiophenes, which are known to be analogous to benzofurans in many cases. The potency, selectivity, and other properties of the analogs can be modified or modulated by inclusion of different substituents appended to the common core structure.

Analogs considered during a redesign effort can be prepared in a number of ways, some of which are known in the art. While compounds can be prepared individually, this tends to be slow and labor-intensive. Combinatorial chemistry techniques have been applied to lead optimization (for references, see, e.g., E. M. Gordon, M. A. Gallop, D. V. Patel. “Strategy and Tactics in Combinatorial Organic Synthesis” Acc. Chem. Res., 1996, 29, 144-154) to generate large numbers of new compounds for screening. Combinatorial techniques presently in use are capable of preparing hundreds or thousands of structurally-related analogs; the analogs can be prepared either in solution phase or attached to a solid support.

In general, techniques of combinatorial chemistry involve the selection of an appropriate reaction scheme for synthesis of a library (or array) of structurally-related compounds, followed by selection of reagents for use in the reaction scheme. The choice of reaction scheme and reagents will together determine the compounds that will ultimately be prepared.

An important consideration in the design of combinatorial libraries or arrays of compounds is the selection of the compounds to be prepared. It is often theoretically possible, using commercially-available reagents, to prepare libraries of hundreds of thousands, or even millions, of compounds. However, the physical preparation of such a large library of compounds is not always practical; in addition to difficulties related to the purchase and handling of reagents, manipulation of such large numbers of reaction mixtures would be a considerable task. It is therefore useful, in accordance with this invention, to select, from the set of compounds theoretically accessible (often referred to as a “virtual library” of compounds), a subset of compounds for synthesis.

Compounds can be selected for synthesis according to several criteria, including: expense of starting reagents required for synthesis; the chemical diversity of compounds selected; and the physical, chemical, or biochemical properties predicted for the compounds (including properties such as mass, solubility, absorption, metabolic stability, toxicity, desired biological activity, and the like). In accordance with this invention, analogs from the virtual library are evaluated for ADMET/PK properties to identify particularly promising candidates.

An initial consideration is the size of the virtual library; if the virtual library is very large, computations may be time-consuming. In accordance with this invention, the size of the virtual library is reduced to “filter” a list of potentially-useful reagents to produce a more manageable list. For example, the reagent list can be filtered to remove reagents having a molecular weight greater than a selected cutoff value (e.g., molecular weight greater than 600 daltons). Similarly, reagents that are highly hydrophobic can also be filtered out, on the assumption that the use of such reagents would be likely to produce compounds that are excessively hydrophobic and, therefore, not drug-like. One additional filter is to eliminate reagents which, if used, would produce compounds having undesired functional groups, such as highly reactive moieties (e.g., haloketones and the like). By using appropriate reagent filters, the numbers of compounds in a virtual library can be reduced.

However, the size of the virtual library may still be too large (even after the application of filters) for computations to be efficiently performed on the compounds of the virtual library. In such cases, some of the properties of the compounds of the virtual library can be approximated based upon the properties of the reagents. For example, the degree of hydrophobicity of a product can be calculated based upon the hydrophobicity of the reagents. For certain properties, such as biological activity, reagent-based calculations are unlikely to be useful. In such cases, it is preferable to make calculations based upon the structure of the products, rather than on the structures of the reagents, although product-based optimizations are more computationally intensive.

Chemical diversity can be measured or calculated for a virtual library of compounds according to methods well-known to the skilled artisan. For example, sets of descriptors for compounds can be generated using methods and computer software that is commercially available (e.g., Cerius2, available from Accelrys (Pharmacopeia, Princeton, N.J.)). These descriptors can then be used to optimize the chemical diversity of the compounds in a subset (of any desired size) of the virtual library. Once the diverse subset is selected, the compounds can be made, e.g., according to the combinatorial methods described above.

Once a compound or library has been prepared, the compound (or library) can be screened to determine whether the compound (or compounds in a library) possess a desired activity (such as a specific biological activity). The compounds can also be screened to determine whether the biological activity, if present, is selective for the desired target, or whether undesirable side effects may occur due to interactions with biological receptors other than the specified target.

Many screening assays are available to the ordinarily-skilled artisan; some are commercially available (e.g., in kit form), while others have been described in the literature. Some screening assays can be performed in a high-throughput mode, which permits screening hundreds or thousands of compounds per day for a particular activity. For example, automated screening platform technology is available from companies chloramphenicol, cipofloxacin, diltiazem, dirithromycin, disulfiram, enoxacin, fluconazole, fluoxetine, fluvoxamine, isoniazid, metronidazole, MAO inhibitors, nefazodone, and trimethoprim/sulfamethoxazole (TMP/SMX).

Many drug-drug interactions involve metabolism. Of particular interest in redesign efforts are compounds implicated in drug-drug interactions that are neither P450 inhibitors nor inducers, but when given in combination with certain P450 inhibitors or inducers, the metabolism of these compounds is so severely affected that they are either ineffective (when given with a P450 inducer) or toxic (when given with a P450 inhibitor). This is because these compounds primarily rely on a single P450 enzyme for their metabolism. If that one P450 is inhibited, these compounds accumulate to toxic levels. Conversely, if that P450 is induced, these compounds are metabolized too quickly and are ineffective. This is why the ideal drug is metabolized by more than just one P450 enzyme. A likely example of a problem caused by a compound being metabolized by only a single P450 enzyme occurred with the cholesterol-lowering drug cerivastatin (Baycol/Lipobay), which Bayer removed from the market. Other, proven, examples include Seldane (terfenadine), Hismanal (astemizole), and Propulsid (cisapride). All three of them were removed from the market because they were implicated in serious drug interactions due to inhibition of their CYP 3A4-mediated metabolism.

The following table contains a listing of drugs susceptible to inhibition or induction of a P450. The numbers in the second columns indicate the number of currently known drug interactions that involve each compound.

TABLE

| | |
|---|---|
| Inhibition | Induction |

| Compound | No. | Compound | No. |
|---|---|---|---|
| acenocoumarol | 1 | acetaminophen | 4 |
| acetophenazine | 1 | alprazolam | 3 |
| alprazolam | 15 | amiodarone | 6 |
| amiodarone | 1 | amitriptyline | 11 |
| amitriptyline | 5 | betamethasone | 15 |
| amoxapine | 3 | bupropion | 1 |
| astemizole | 10 | buspirone | 2 |
| atorvastatin | 5 | carbamazepine | 9 |
| bepridil | 1 | chlordiazepoxide | 3 |
| buspirone | 3 | clarithromycin | 1 |
| carbamazepine | 3 | clomipramine | 11 |
| cerivastatin | 5 | clonazepam | 3 |
| chlordiazepoxide | 7 | clorazepate | 3 |
| chloroquine | 1 | clozapine | 1 |
| chlorpromazine | 1 | corticotropin | 9 |
| chlorzoxazone | 1 | cortisone | 15 |
| cisapride | 15 | cosyntropin | 20 |
| clarithromycin | 1 | cyclosporine | 6 |
| clomipramine | 5 | delavirdine | 2 |
| clonazepam | 10 | desipramine | 1 |
| clorazepate | 7 | detromethorphan | 1 |
| clozapine | 2 | dexamethasone | 14 |
| codeine | 1 | diazepam | 3 |
| cyclosporine | 7 | disopyramide | 3 |
| desipramine | 4 | doxepin | 11 |
| diazepam | 14 | doxycycline | 3 |
| dihydroergotamine | 7 | erythromycin | 1 |
| doxepin | 4 | estazolam | 3 |
| encainide | 2 | felodipine | 4 |
| ergotamine | 7 | fludrocortisone | 14 |
| estazolam | 7 | flurazepam | 3 |
| felodipine | 1 | halazepam | 3 |
| fentanyl | 5 | haloperidol | 3 |
| flecainide | 2 | hydrocortisone | 14 |
| fluphenazine | 1 | imipramine | 11 |
| flurazepam | 7 | indinavir | 3 |
| fluvastatin | 1 | itraconazole | 5 |
| halazepam | 7 | ketoconazole | 2 |
| hydrocortisone | 1 | mephenytoin | 2 |
| imipramine | 5 | methadone | 8 |
| indinavir | 2 | methylprednisolone | 14 |
| lidocaine | 2 | miconazole | 2 |
| lovastatin | 5 | midazolam | 3 |
| mephenytoin | 4 | nelfinavir | 2 |
| mesoridazine | 1 | nifedipine | 9 |
| methotrimeprazine | 1 | nisoldipine | 4 |
| methylprednisolone | 1 | nortriptyline | 11 |
| metoprolol | 3 | oral contraceptives | 33 |
| midazolam | 15 | phenobarbital | 1 |
| nifedipine | 1 | phenytoin | 1 |
| nisoldipine | 3 | prednisolone | 10 |
| nortriptyline | 4 | prednisone | 10 |
| omeprazole | 1 | quazepam | 3 |
| oral contraceptives | 1 | quinidine | 13 |
| perphenazine | 1 | quinine | 2 |
| phenprocoumon | 1 | ritonavir | 3 |
| phenytoin | 4 | tacrolimus | 3 |
| pimozide | 3 | tamoxifen | 2 |
| piroxicam | 1 | toremifene | 3 |
| pravastatin | 1 | triamcinolone | 10 |
| prazapam | 2 | triazolam | 3 |
| prednisolone | 1 | trimipramine | 9 |
| prednisone | 1 | troleandomycin | 1 |
| prochlorperazine | 1 | verapamil | 1 |
| promazine | 1 | warfarin | 2 |
| promethazine | 1 | zolpidem | 2 |
| propafenone | 1 | | |
| propiomazine | 1 | | |
| propoxyphene | 1 | | |
| propranolol | 2 | | |
| protriptyline | 3 | | |
| quazepam | 7 | | |
| quinidine | 4 | | |
| rifabutin | 6 | | |
| rifampin | 2 | | |
| rifapentine | 2 | | |
| ritonavir | 2 | | |
| saquinavir | 2 | | |
| sildenafil | 5 | | |
| simvastatin | 5 | | |
| tacrolimus | 4 | | |
| terfenadine | 10 | | |
| thiethylperazine | 1 | | |
| thioridazine | 2 | | |
| timolol | 1 | | |
| tolbutamide | 1 | | |
| triazolam | 15 | | |
| trifluoperazine | 1 | | |
| triflupromazine | 1 | | |
| trimipramine | 4 | | |
| valproic acid | 1 | | |
| vinblastine | 4 | | |
| vincristine | 4 | | |
| warfarin | 12 | | |
| zolpidem | 2 | | |

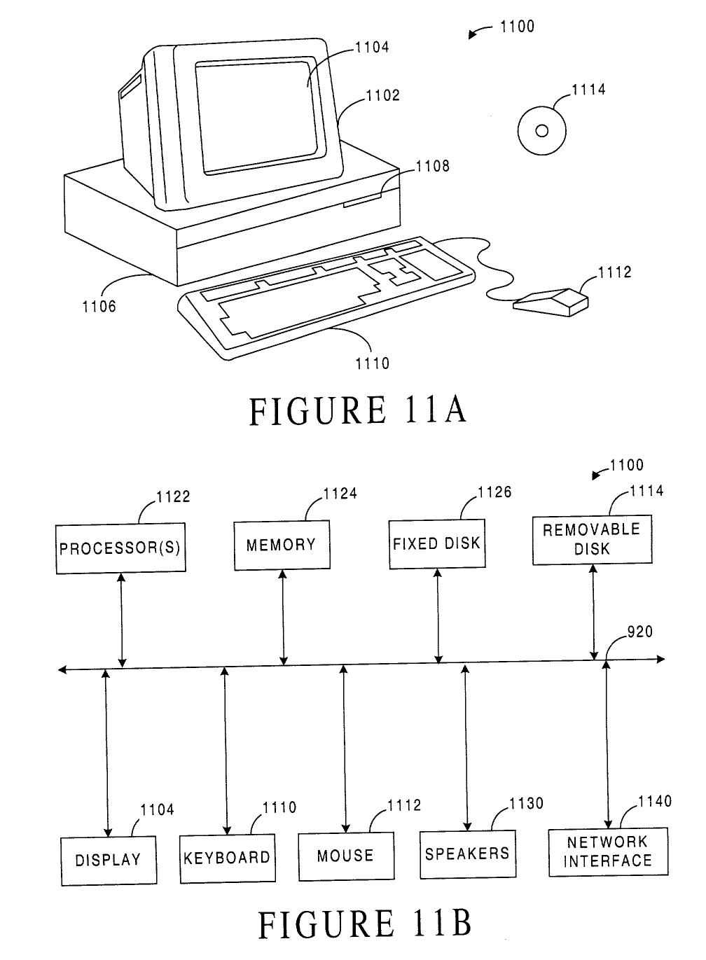

Another class of compounds that can be considered for drug rescue includes therapeutic compounds that are cytochrome P450 inhibitors. These are compounds that cause more or less serious problems when co-administered with certain other drugs. The interaction is due to their ability to bind tightly to specific P450 enzymes and to inhibit the enzyme's ability to metabolize the co-administered drug, which in turn drug discovery site, for example, sends data identifying the therapeutic compounds 1208 to a processing server, 1206 via the Internet 1204. At the processing server 1209, the therapeutic compounds are analyzed by a model 1212, which analyzes the therapeutic compound in accordance with the current invention. As mentioned, such model may take the form of a software application or a suite of software tools, for example. As part of the model, or as provided by a separate software program or hardware, the server may include logic for generating a detailed structural representation of a chemical compound from the identity of the chemical compound or from course structural information. Using the available software or other logic, the server generates one or more redesigned therapeutic chemical compounds. After the analysis, the redesigned compounds 1210 are sent via the Internet 1204 back to the client 1202. The computer system illustrated in FIGS. 11A and 11B is suitable both for the client 1202 and the processing server 1206. In a specific embodiment, standard transmission protocols such as TCP/IP (transmission control protocol/internet protocol) are used to communicate between the client 1202 and processing server 1206. Custom or standard security measures such as SSL (secure socket layer), VPN (virtual private network) and encryption methods (e.g., public key encryption) can also be used.

Although certain details have been omitted for brevity's sake, various design alternatives may be implemented. Therefore, the present examples are to be considered as illustrative and not restrictive, and the invention is not to be limited to the details given herein, but may be modified within the scope of the appended claims. 10A that analogs 1009, 1011, 1013, and 1015 with log BBB values greater than zero are predicted to breach the BBB.

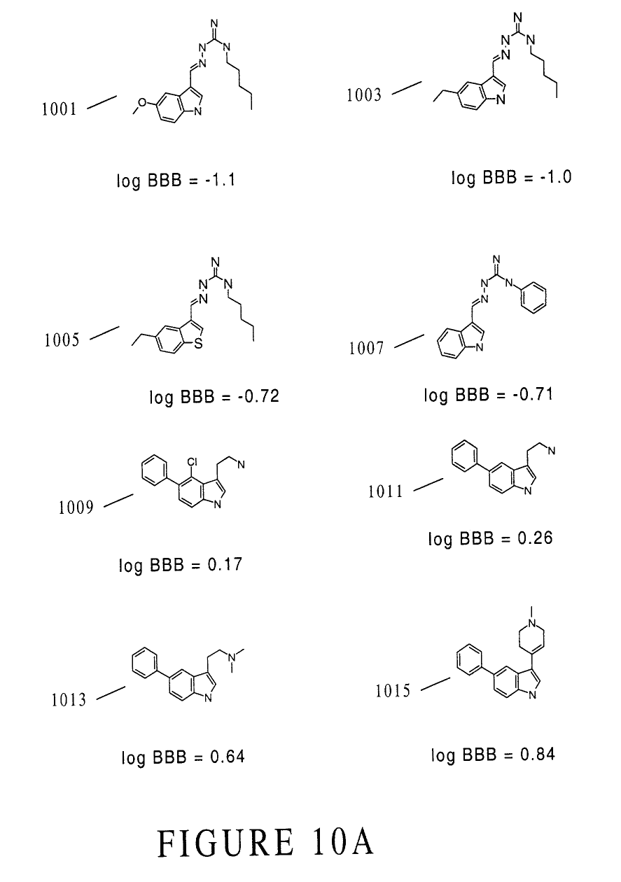

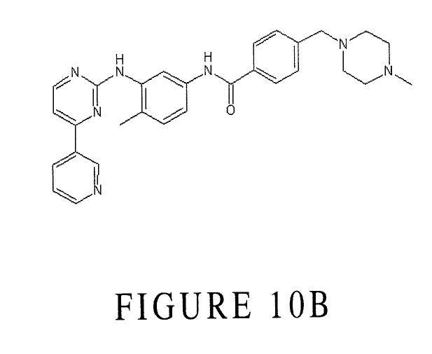

The ability to act on the CNS of imatinib (brand name Gleevac®) was also predicted. Imatinib is an Bcr-Ab1 tyrosine kinase inhibitor that has been approved for chronic myelogenous leukemia and is under investigation to treat glioblastomas. The chemical structure of imatinib is shown in FIG. 10B. The log BBB value of imatinib was predicted to be −0.65, which correctly shows that imatinib has a low probability of breaching the BBB and acting on the CNS.

Hardware and Software Implementation of the Invention

Generally, embodiments of the present invention employ various processes involving data stored in or transferred through one or more computer systems. The methods of this invention may be implemented in whole or in part using such computer systems. Embodiments of the present invention also relate to an apparatus for performing these method operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

FIGS. 11A and 11B illustrate a computer system 1100 suitable for implementing embodiments of the present invention. FIG. 11A shows one possible physical form of the computer system. Of course, the computer system may have many physical forms ranging from an integrated circuit, a printed circuit board and a small handheld device up to a huge super computer depending on the processing requirements of the embodiment. Computer system 1100 includes a monitor 1102, a display 1104, a housing 1106, a disk drive 1108, a keyboard 1110 and a mouse 1112. Disk 1114 is a computer-readable medium used to transfer data to and from computer system 1100.

FIG. 11B is a block diagram of the architecture of computer system 1100. Attached to system bus 1120 are a wide variety of subsystems. Processor(s) 1122 (also referred to as central processing units, or CPUs) are coupled to storage devices including memory 1124. Memory 1124 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable of the computer-readable media described below. A fixed disk 1126 is also coupled bi-directionally to CPU 1122; it provides additional data storage capacity and may also include any of the computer-readable media described below. Fixed disk 1126 may be used to store programs, data and the like and is typically a secondary storage medium (such as a hard disk) that is slower than primary storage. It will be appreciated that the information retained within fixed disk 1126, may, in appropriate cases, be incorporated in standard fashion as virtual memory in memory 1124. Removable disk 1114 may take the form of any of the computer-readable media described below.

CPU 1122 is also coupled to a variety of input/output devices such as display 1104, keyboard 1110, mouse 1112 and speakers 1130. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 1122 optionally may be coupled to another computer or telecommunications network using network interface 1140. Interface 1140 may operate under one or more network protocols (at various levels) such as Ethernet, frame relay, ATM, cable protocols, fiber optical network protocols, wireless protocols, etc. With such a network interface, it is contemplated that the CPU might receive information from the network, or might output information to the network in the course of performing the above-described method operations. Furthermore, method embodiments of the present invention may execute solely upon CPU 1122 or may execute over a network such as the Internet in conjunction with a remote CPU that shares a portion of the processing.

In addition, embodiments of the present invention further relate to computer storage products with a computer-readable medium that have computer code thereon for performing various computer-implemented operations. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), ROM and RAM devices, and signal transmission media for delivering computer-readable instructions, such as local area networks, wide area networks, and the Internet. Examples of computer code include source code, machine code, such as produced by a compiler, and files containing higher level code that are executed by a computer using an interpreter. The invention also pertains to carrier waves and transport media on which the data and instructions of this invention may be transmitted.

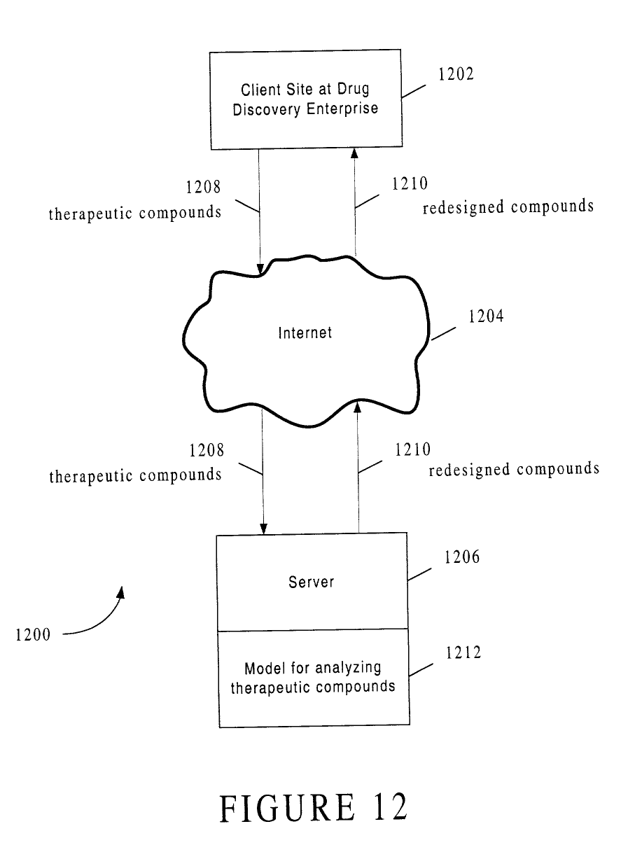

In one embodiment, the computer system 1100 is directly coupled to an external source, such as the Internet (see FIG. 12) that provides the identity of various drug rescue candidates. Chemical compounds identified from external sources are provided via network interface 1140 or other I/O interface such as keyboard 1110. In one example, the compounds analyzed by system 1100 are provided from a storage source such as a database or other repository of chemical information. Again, the chemical compound identities are provided via interface 1140. Once in the computational apparatus 1100, a memory device such as primary storage 1124 or mass storage 1126 buffers or stores, at least temporarily, the identities and other information about the chemical compounds.

Optionally, computer system 1100 can store and/or execute instructions that generate detailed structural information (described above) about chemical compounds from the identities of the chemical compounds or from less detailed structural information. Typically, structural information about the chemical compounds (whether obtained from an external source or generated internally) can be analyzed by program instructions executing on processor 1122 to identify sites that may be responsible for less than optimal ADMET/PK properties. Such properties may include one or two or more of the following: absorption, metabolism, distribution, excretion, and toxicity (see page 12). Further, the processor 1122 may execute instructions that allow it to specify a modification to the chemical compound in order to modify at least one of the following properties of the compound: absorption, metabolism, distribution, excretion, and toxicity (see page 12). The instructions executing on processor 1122 may come from various sources. For example, they may be stored within system 1100 on memory 1124 and/or memory 1126. The instructions may also come from an external source such as a network source via interface 1140. Alternatively, the processor itself may include firmware that provides the instructions or the processor may have a hardware design for automatically performing some operations required to perform the analysis.

FIG. 12 is a schematic illustration of an Internet-based embodiment of the current invention. See 1200. According to a specific embodiment, a client 1202, at a such as Aurora Biosciences (San Diego, Calif.). Such high-throughput screening assays are useful for rapidly screening combinatorial libraries which may include thousands of compounds.

Examples of assays which can be used to screen compounds include (but are not limited to) the following: assays for inhibition of a serine protease (see, e.g., Seymour et al. (1994) Biochemistry 33:2949-2958 and Nagahara et al., (1994) J. Med. Chem. 37:1200-1207); assays for inhibition of a cysteine protease (see, e.g., Cazzulo et al. (1990) Biochim. Biophys. Acta 1037:186-191); assays for protein-protein interactions (for references to scintillation proximity assay (SPA), see, e.g., Scheffler et al. (1996) Techniques in Protein Chemistry VII, Academic Press, pp. 101-106); and Gorman et al, (1996) J. Biol. Chem. 271:6713-6719); and assays for metalloprotease inhibition (see, e.g., Knight et al., FEBS Lett. (1993) 296:263-266). In many cases, the results of such assays can be provided in tabular form, corresponding to, e.g., 96-well plates in which the assays were performed; compounds which show a desired activity can be identified from the tabular report and identified by determining which compound was present in the corresponding position of the 96-well plates.

Screening compounds for therapeutic activity, to select lead compounds, can also be accomplished using “virtual screening” of compounds (that is, computer-based calculation of compound properties in silico, rather than in vitro). Such in silico screening has the advantage that no chemical reagents or assay equipment is required, which reduces cost. Another advantage of virtual; screening is that chemical interferences with assay performance (e.g., interfering substances present in a chemical assay system which may cause false positive or false negative results) are eliminated.

However, virtual screening does require careful selection of the computer models employed for screening. In preferred embodiments, the parameters upon which a computer model is premised can be adjusted or re-parameterized, e.g., by comparing the results of virtual screening with assay result obtained from in vitro screening for the same property, followed by correction of the in silico model. Such reparameterization can also be applied to ADMET/PK models employed with this invention—as appropriate.

### EXAMPLES

Fluoxetine (brand name drug Prozac®) was identified as a therapeutic chemical compound for rescue due as a cytochrome P450 inhibitor. Fluoxetine binds tightly to the CYP2D6 enzyme, which inhibits the enzyme's ability to metabolize certain drugs if they are co-administered with fluoxetine. As a result, the co-administered drug accumulates in the body to toxic levels and causes harm. Fluoxetine was analyzed utilizing software to analyze various of its properties, including metabolism. In addition, various modifications to fluoxetine were specified in order to modify the compound's metabolism.

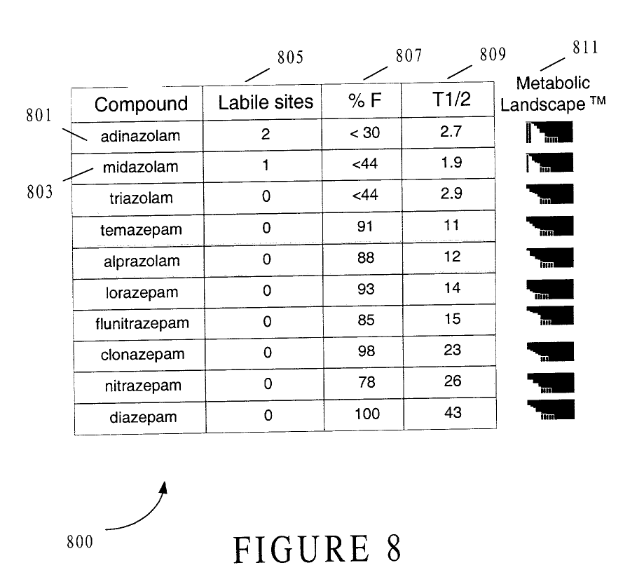

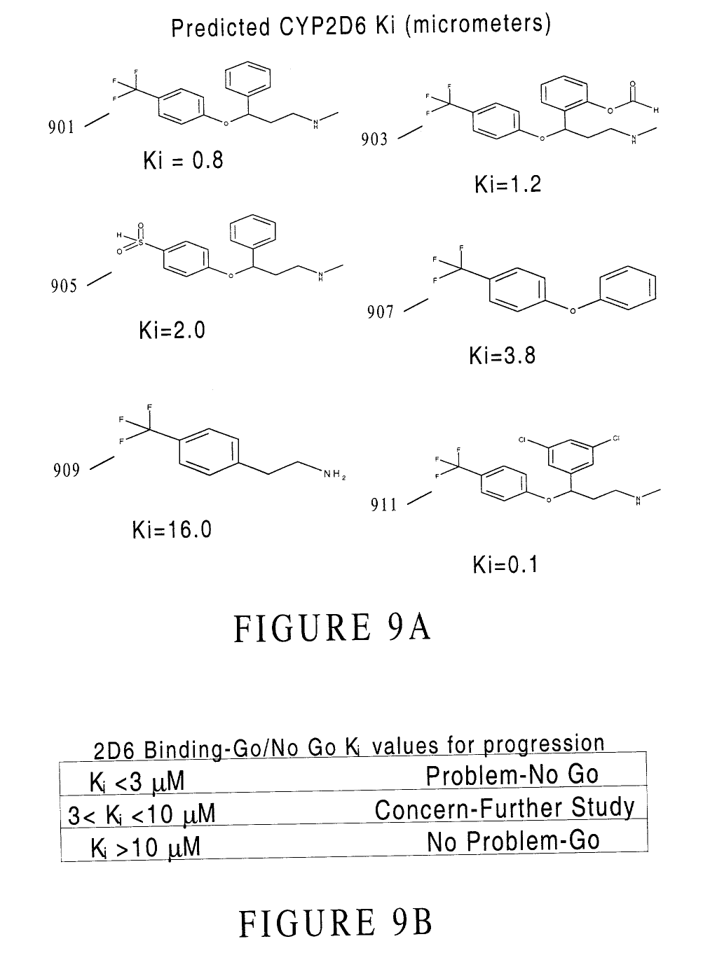

FIG. 9A shows the structure of fluoxetine 901 and five chemicals that were identified as potential rescue analogs. See 903, 905, 907, 909, and 911. The potential rescue analogs were specifically generated for this example.. Since the inhibitory effect is caused by fluoxetine's binding affinity (Kᵢ) to the CYP2D6 enzyme, the binding affinities of all the potential rescue analogs were also computed to determine if the potential rescue analog would also exhibit an inhibitory effect or not. The lower the value of K₁, the tighter the molecule binds to CYP2D6. FIG. 9B shows possible ranges of binding affinities for which CYP2D6 inhibition may be a problem.

As seen in FIG. 9B, in this example potential rescue analogs with K₁ less than 3 μm are considered to be CYP2D6 inhibitors, and so are not worth further study. A K₁ greater than 10 μm indicates that the potential rescue analog will not be an inhibitor, while analogs with predicted Kᵢs between 3 and 10 μm require further study to determine if they should be construed as CYP2D6 inhibitors. The binding affinity Kᵢ of each analog to the CYP2D6 enzyme was predicted using a descriptor-based model built with an internally generated inhibition data set and multivariate statistics. FIG. 9A also shows the binding affinities as predicted by the model. It can be seen from FIG. 9A that the model predicted that the potential rescue analog 909 would not exhibit the CYP2D6 inhibitory effects and that potential rescue analog 907 requires further study according to the ranges shown in FIG. 9B. The other analogs are predicted by the model to be CYP2D6 enzyme inhibitors and so do not merit further investigation as potential rescue analogs to fluoxetine. The model correctly predicted that fluoxetine is a CYP2D6 inhibitor with a Kᵢ of 0.8 μm. The experimental value of Ki for fluoxetine is 0.3 μm (Pfizer Corp., ACS Meeting, San Diego, Calif. Apr. 4, 2001).

The therapeutic chemical compound tegaserod (brand name drug Zelmac®) is a 5-HT receptor agonist that is FDA approved for irritable bowel syndrome in women. It was identified as a compound for rescue because development of the drug for memory enhancement was abandoned when it was found not to breach the Blood-Brain-Barrier (BBB). Tegaserod was analyzed utilizing software to analyze various of its properties, including ability to penetrate the blood brain barrier. In addition, various modifications to tegaserod were specified in order to modify the compound's BBB penetration ability.

FIG. 10A shows the structure of tegaserod 1001 and seven chemicals that were identified as potential rescue analogs. See 1003, 1005, 1007, 1009, 1011, 1013, and 1015. Analogs 1003, 1005, and 1007 were designed for this example. Analogs 1009, 1011, 1013, and 1015 were identified as potential rescue analogs within the MDDR database (the MDL Drug Data Report, 2001 edition). Log BBB, the log of the partition ratio between the brain tissue and the plasma, was predicted for tegaserod and the potential rescue analogs using a model built using multivariate statistics and a permeability model.

More specifically, the model employed in this example predicts the log of the partition ratio (of brain to blood) at the blood-brain barrier (BBB) for compounds with a molecular weight greater than 200 (“drug-like” compounds). A value of 0 means the compound is equally distributed in the blood and the brain compartments. A value of −1 means the ratio of the concentrations in the brain to the blood compartments is one-tenth.

A blood-brain partition model was developed using literature data of 45 compounds in rodents (Kelder, J., et al., Pharm. Res. 16: 1514-1519, 1999), with molecular weights from 204 to 450. The range of hydrogen bond acceptors (HBA) was from 0 to 6. The range of hydrogen bond donors (HBD) was from 1 to 13.

The linear partial least squares (PLS) model identified three latent variables or principal components, representing more than 75% of the X-variables. The most important descriptors are hydrogen bond and molecular surface area related properties. The model was externally validated using 26 compounds different from the training set, lying within the 90% confidence intervals of the chemical-structure spaces established by the first few principal components in the X-spaces, taken from the literature (Luco, J. M., J. Chem. Inf. Comput. Sci. 39[2]: 396-404, 1999). The square of the correlation coefficient was 0.76, and the predicted root mean square error was 0.42.

The model was also externally validated using 95 compounds able to cross the blood-brain barrier, obtaining a score of 100% when the predicted log BB was larger than −0.75. The model was also validated using 135 compounds with little ability to cross the blood-brain barrier, with scores of 78% when the predicted log BB was smaller than −0.5 (Crivori, P., et al., J. Med. Chem. 43[11]: 2204-2216, 2000).

FIG. 10A shows the predicted log BBB values of tegaserod and the analogs. The BBB model employed with the present invention determined a log BBB of −1.1 for tegaserod, correctly predicting that it does not act on the CNS. It can be seen from FIG.

P-gp is an integral membrane protein. It is made up of two homologous halves, each consisting of an N-terminal hydrophobic domain with six transmembrane segments that is separated from a hydrophilic domain containing a nucleotide-binding fold by a flexible linker polypeptide. P-gp's efflux action is powered by ATP-hydrolysis. Other active transport proteins that are modeled according to the current invention include MDR2 and cMRP (also known as cMOAT or MRP2) that translocate phospholipids, glutathione, glucuronide, and sulfate conjugates of certain drugs across the hepatocyte canalicular membrane and monocarboxylic acid transporter MCT1, which mediates the bi-directional transport of some organic anions at the Blood-Brain-Barrier (BBB) and the efflux of valproic acid and probenecid.

A striking feature of P-pg and these other transporters are their broad substrate specificities. P-gp transports many structurally dissimilar drugs that act on different intracellular targets, including anticancer drugs, cardiac drugs, anti-fungal and anti-microbial agents, immunosuppressive agents, HIV protease inhibitors, steroids, calcium channel blockers, and other cytotoxic drugs. While not entirely non-specific, these proteins do appear to be highly “versatile” in their substrate interactions.

As discussed above, an important consequence of this versatility is that the computational tools in the current art used for the modeling of classical protein-substrate interactions are not useful for modeling these active transporters. Ordinary modeling of selective protein interactions specifically focuses on the atomic interactions that impart the specificity. Likewise, pharmacophore-based approaches and field-based approaches use the shared three-dimensional characteristics of ligands (and non-ligands) to construct a three-dimensional representation of the binding site.

In a preferred embodiment, the invention employs at least some descriptor-based structure-activity relationship (SAR) models that are based on the physical and chemical properties of the substrates alone and that disregard the structure of the substrate-binding fold of the transporter. These descriptors represent individual or composite molecular properties and are used to relate structure to activity. They include, but are not limited to, atomic and functional groups-based descriptors, such as the number of aromatic halides, amides, or basic nitrogens, and specific fragment descriptors. Again see U.S. patent application Ser. No. 09/811,283 (Atty Docket No.: CAMIP005) for further discussion of such descriptors. They also include whole molecule property descriptors, such as lipophilicity, size, and HOMO/LUMO (Highest Occupied Molecular Orbital and Lowest Unoccupied Molecular Orbital) descriptors.

Modeling of active transport-mediated and passive transport independent movement of drug compounds across cell membranes is carried out in a similar manner to the intestinal processes described above. Aqueous solubility is also an important component of distribution that is taken into account by the current invention.

Distribution and Excretion

As mentioned, modeling of ADMET/PK properties can include modeling distribution and/or excretion or particular compounds. “Distribution” includes Blood-Brain-Barrier (BBB) penetration, protein binding in the blood, receptor-binding and drug transport across cell membranes. The active and passive transport models are similar to those discussed above with respect to absorption models. Modeling of excretion includes hepatic and renal excretion, which are also mediated by active and passive transport mechanisms, and require similar models. For instance, P-gp is expressed on the brush border and biliary face of proximal tubule cells in the kidney and hepatocytes, respectively, consistent with a role for this active transporter in the excretion of compounds into the urine and bile.

In general, distribution and excretion can be modeled as described above, preferably using a collection of suitable structural descriptors. The descriptors identify key structural motifs that strongly impact a distribution and/or excretion mechanism.

Toxocity Analysis and Redesign

The effect of toxic compounds involves all aspects of their interaction with the biological system, including absorption, distribution, metabolism, elimination, and other biochemical reactions. Metabolism, in particular, plays a complex role in toxicity as it serves both as a mechanism for detoxification and as a process that can create toxic compounds. Detoxification is achieved through the metabolism and removal of exogenous compounds from the biological system. Compounds that are not metabolized rapidly enough or whose metabolism is inhibited by the co-administration of another drug may build up to toxic levels and lead to adverse effects. Besides this detoxification function, metabolism can also cause toxicity problems by transforming harmless compounds into more reactive molecules that are toxic. For example, the oxidation reaction of acetaminophen that is catalyzed by CYP 2E1 and CYP 3A4 produces electrophilic metabolite(s). Similarly, a compound's rate of absorption, distribution, and elimination can have important implications for its toxicity profile. For example, neurotoxicity is likely to be low for compounds that cannot cross the blood-brain barrier. Genetic polymorphisms in metabolism enzymes and transporter molecules may also affect the local concentration of compounds and their metabolites at target sites and can be at the root of variations in toxicity profiles observed across patient populations.

Computational models in the current art for predicting toxicity can, for example, generally be categorized as either (1) knowledge-based or (2) statistically-based. Knowledge-based approaches codify human expert judgment into generalized rules that can be used to predict the toxicity endpoints of novel compounds. They do not discover new associations, but rather attempt to mimic human expert reasoning derived from past experience. Knowledge-based approaches were used to generate commercial toxicity programs such as OncoLogic, DEREK, and HazardExpert. Statistically-based approaches use databases of toxicity date and data mining techniques to uncover associations between chemical structures and biological activities. The structures are encoded with molecular descriptors, the distribution of which among active and inactive molecules in the training set is analyzed statistically. Molecular descriptors that are strongly associated with specific toxicity endpoints are incorporated into predictive, computational models. Statistically-based approaches used in commercial programs such as CASE, MULTICASE, and TOPKAT.

The major limitation of knowledge-based approaches relates to inadequate rules for a number of toxicity endpoints and chemical classes due to knowledge gaps of the expert developers. Statistically-based approaches, since they do not rely on expert knowledge they can be more broadly applicable. However, since most chemical toxicity databases contain compounds whose toxicities are mediated by a diverse range of biological mechanisms, detection of strong associations between molecular descriptors and toxicity endpoints is a challenging proposition. Subdivision of these databases into specific toxicity pathways generally results in significantly smaller databases. Computational models derived from these smaller databases tends to be applicable to only a limited range of compounds in a narrow range of chemical space. Therefore, the quality and applicability of statistically-based prediction models is critically dependent on the use of large databases of highly diverse compounds with specific toxicity endpoints.

The computational model of the current invention involves the used of the two million adverse drug events recorded over the past 32 years through the Adverse Event Reporting System (AERS) by the FDA Center for Drug Evaluation and Research (CDER). This information was collected in the Adverse Event database, a computerized

In another approach, the descriptors are molecular fragments. These are stored with associated values of the ADMET/PK property of interest. The ADMET/PK values obtained by either approach may be corrected with correction factors such as steric correction factors. In one embodiment, the ADMET/PK property values of the fragments obtained from a training set can be stored in a database. If a statistically significant number of reactivities have been computed for a given fragment, and the variance of the values about a mean are within an accepted threshold, then the fragment reactivity is trusted and may be used with confidence.

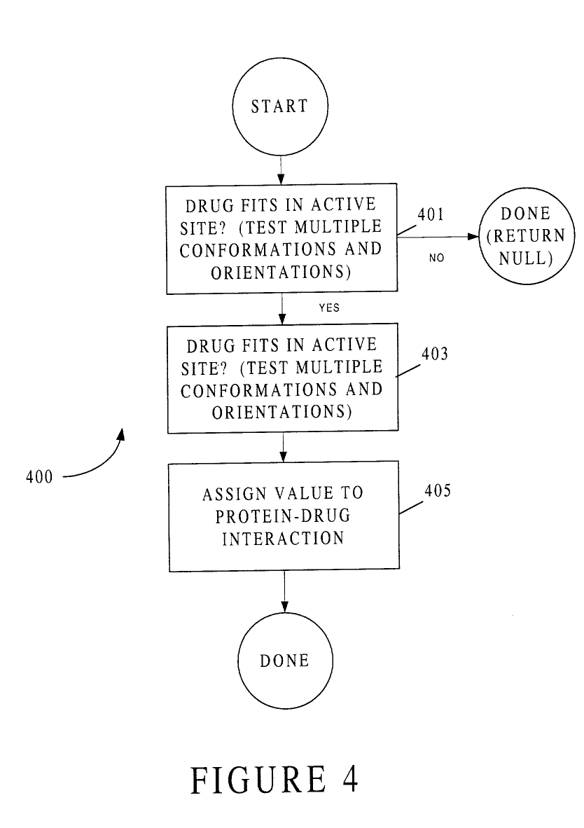

One example of how these descriptors are used is in the context of active-transport modeling, other protein-drug binding interactions. Active transport is a critical component of drug absorption and distribution. FIG. 4 illustrates from a high-level, a computer-implemented process of the current invention for modeling such interactions. See 400. First, the process determines if the drug molecule being analyzed can fit at all in the active site of the protein. See 401. The molecule is tested in multiple conformations and orientations. If not, then the process returns a null value, indicating that this protein-mediated process (e.g., active transport) is a not a factor in the ADMET/PK behavior of the drug. If there is some possibility of protein-drug interaction, then the process will apply the descriptor model to the relevant regions of the compound. See 403. Using the descriptor model, a quantitative value is this assigned to the protein-drug interaction, typically an activation energy value that is determined by summing all the relevant interactions. See 405. The importance of the interaction to the overall property being analyzed can thus be determined. Some of the proteins that can be subjected to this kind of analysis include the CYP proteins, the active transport proteins P-glycoprotein, MCT1, MDR2 and cMRP (also known as cMOAT or MRP2), and human serum albumin and other binding proteins.

Metabolic Analysis and Redesign

A large portion of all drug metabolism in humans and most all organisms is carried out by the cytochrome p450 enzymes. The cytochrome p450 enzymes (CYP) are a superfamily of heme-containing enzymes that include more than 700 individual isozymes that exist in plant, bacterial and animal species. Nelson et al. Pharmacogenetics 1996 6, 1-42. They are monooxygenase enzymes. Wislocki et al., in Enzymatic Basis of Detoxification (Jakoby, Ed.), 135-83, Academic Press, New York, 1980. Although humans share the same several CYP isozymes, these isozymes can vary slightly between individuals (alleles) and the isozyme profile of individuals, in terms of the amount of each isozyme that is present, also varies to some degree. It is estimated that in humans, 50% of all drugs are metabolized partly by the p450 enzymes, and 30% of drugs are metabolized primarily by these enzymes. The most important CYP enzymes in drug metabolism are the CYP3A4, CYP2D6 and CYP2C9 isozymes. Although limited success has been achieved with the modeling of some enzyme metabolism, the CYP enzymes are especially difficult to model because of their non-specificity. For a further discussion of CYP enzyme modeling, see U.S. patent applications Ser. No. 09/368,511 (Atty Docket No.: CAMIP001), Ser. No. 09/613,875 (Atty Docket No.: CAMIP002), and Ser. No. 09/811,283 (Atty Docket No.: CAMIP005). Because of the importance of CYP enzyme to drug metabolism, CYP enzyme modeling is a critical component of redesign and rescue of drugs.

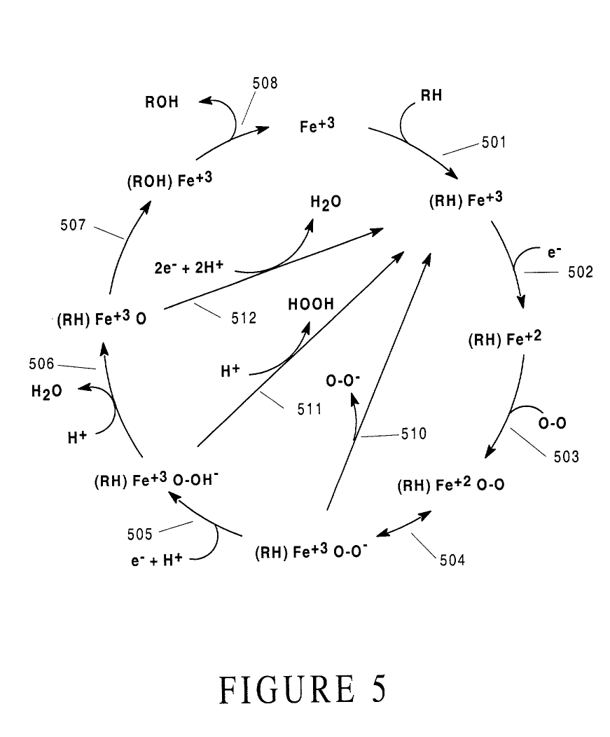

FIG. 5 illustrates the oxidative hydroxylation catalytic cycle for the mammalian CYP enzyme. The top of the figure shows a generic starting substrate (RH) and generic product (ROH). This hydroxylation reaction is often the first step in metabolizing an exogenous compound, and partly explains the importance of the CYP enzymes in drug deactivation/metabolism. For further discussion of the CYP catalytic cycle, see U.S. patent application Ser. No. 09/613,875 (Atty Docket No.: CAMIP002). Other enzymes involved in metabolism include uridine-diphosphate glucuronic acid glucuronyl transferases and glutathione transferases. The present invention may employ models for any of a number of different metabolic enzymes.

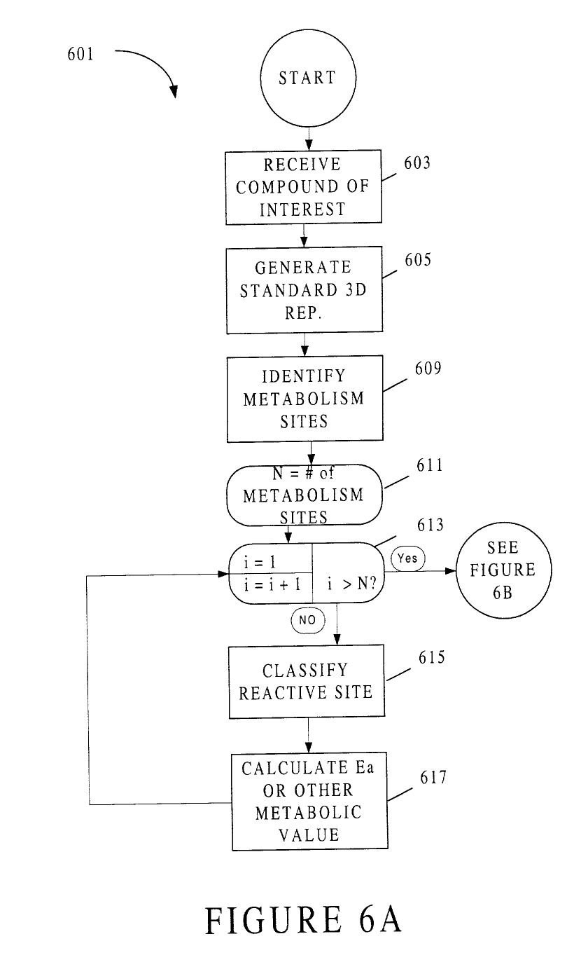

FIG. 6A and 6B together make up a flowchart describing one model for analyzing the metabolic properties of a therapeutic compound as part of the redesign process. Initially, the identity of the compound under consideration is received at an operation 603. Molecular structural information required for the model is either received with the compound identity or generated at an operation 605. The needed structural information can take the form of structural descriptors, fragments, or full three-dimensional structures for example. If a simple look up operation or simple mathematical equation is used, the structural information will often take the form of fragments or structural descriptors as described above. If the model is a quantum chemical model, then the structural information must be a detailed three-dimensional representation of the compound. In all cases, the computational system may receive the structural information as an organic chemistry string of atoms, a two-dimensional structure, a IUPAC standard name, a 3D coordinate map, or as any other commonly used representation. If not already in 3D form, and such form is required by the model, a 3D coordinate map of the molecule is generated, using a geometry program such as Corina or Concord. This approximate 3D geometry structure is then typically optimized with a more sophisticated modeling tool, typically AM1. AM1 is a semi-empirical quantum-chemical modeling program that optimizes the given 3D structure to that local energy minimum. As mentioned above, it calculates electron density distributions from approximate molecular orbitals. It also calculates an enthalpy value for the molecule.

The process then identifies each reactive site of metabolism on the molecule. See 609. The process then analyzes each reactive site, beginning with operations 611 and 613, where the system sets a variable N equal to the number of reactive sites to be considered (611) and iterates over those sites (611). Iterative loop operation 613 initially sets an index value “i” equal to 1. It then determines whether the current value of i is greater than the value of N. If not, it performs various operations to determine the activation energy (E_{A}) at that site.

Analysis of the reactive sites can be carried out from a quantum chemical approach. Quantum chemical approaches are highly accurate when compared to empirical measurements (to within 0.1 kcal/n or less at each reactive site) but are computationally intensive. Some quantum chemical approaches to analyzing the metabolic properties of a compound are described in U.S. patent application Ser. No. 09/613,875 by Korzekwa et al. (Atty Docket No.: CAMIP002). Non-quantum chemical approaches, such as fragment-based or geometry-based approaches that use structural descriptors to describe the metabolic properties of a compound are less accurate (typically to within 0.5 to 1.5 kcal/n) but are much faster computationally. Non-quantum chemical approaches are typically used in situations where high throughput is desirable. Some of these approaches are described U.S. patent application Ser. No. 09/811,283, Ewing et al. (Atty Docket No.: CAMIP005). A combination of quantum chemical and non-quantum chemical approaches can also be used, as described in the same reference.

Regardless of the approach used, the process will typically classify the reactive site in one of several appropriate categories based on its structure (e.g., aromatic oxidation site). See 615. An activation energy value or other metabolic descriptor is then calculated. See 617. When i is greater than N, indicating that all the reactive sites have been analyzed, the process outputs a regioselectivity table or other arrangement of data that indicates the relative lability and activation energies of each of the reactive sites. See 619.

Next, it is useful, though not necessary, to map the reactive sites to a user output relative rates curve based on their activation energies or other metabolic descriptors. See 621. The reactive sites are then binned based upon these relative rates. See 623. art may be employed to filter likely inefficacious compounds, including empirical assays, preferably those with high-throughput, that are well-known to those of skill in the art. Other techniques, automatic and manual, are well-known to those of skill in the art.

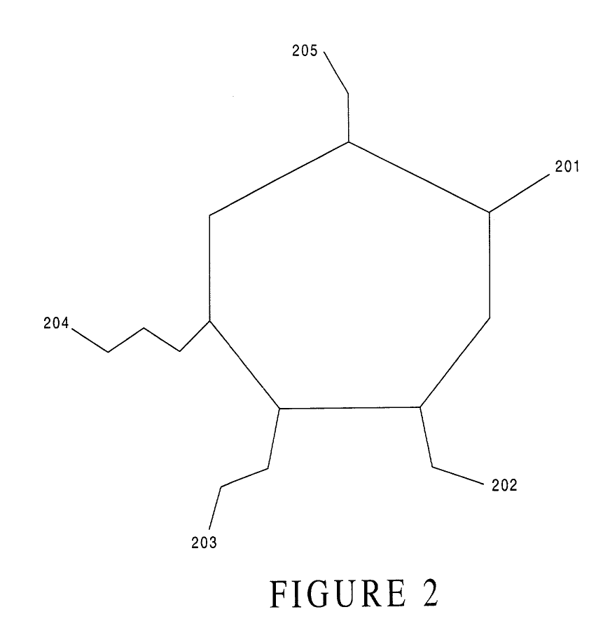

In a typical case, the invention will encounter a compound having various functional sites, each of which can potentially effect the overall ADMET/PK properties of the compound. FIG. 2 is a simplified, schematic illustration of a therapeutic molecule with various functional sites, 201-205, that contribute to the ADMET/PK properties of the molecule. Any one site may be polar (amine, hydroxyl, carbonyl, etc.), reactive (vinyl, ester, etc.), stable, hydrophilic, hydrophobic, etc. Multiple of these sites may together contribute to a single ADMET/PK property of the compound. By choosing any single site for modification, without considering additional sites, a designer may inadequately redesign the compound.

One of the most common ADMET/PK problems with a drug candidate is that it is metabolized too quickly. In many cases, an ideal drug would be metabolized slowly enough so that it can be administered about once a day. In the current art, if a drug candidate was being metabolized too quickly for daily administration, the designers of the drug would try to redesign it, typically by modifying the most reactive site in a manner that would make it considerably more stable.

But changing any one site identified as problematic, may or may not result in an appreciable improvement in the overall ADMET/PK properties of the compound. The result is essentially unpredictable by methods of the current art. A drug designer much less has the ability to predict how a more minor change in a functional site will affect the ADMET/PK properties of the drug. For instance, site 203 might be observed to be the most reactive site. A drug designer could then modify it to make it more stable or even unreactive in an attempt to decrease the overall metabolic rate of the substrate. In some instances this will be successful, but if the substrate has one or more reactive sites that also have relatively high reactive rates, then these sites will often “take over” the metabolism of the substrate and the overall metabolic rate will remain essentially unchanged.

Therefore, a drug designer would have to go through the time-consuming process of redesigning one site as essentially a shot in the dark, re-testing the ADMET/PK properties, and then redesigning that site and/or one or more of the other reactive sites as additional shots in the dark. After conducting this process on most or all of the reactive sites of the drug, the designer might find that it is essentially impossible to achieve the ADMET/PK properties that are desired, particularly without reducing, or perhaps destroying, the desired pharmocological properties of the drug. The chances of altering the pharmocological properties of the drug greatly increase as more and more redesigns of the drug are carried out.

The methods and apparatus of the present invention may account for these multiple site effects by separately considering each site to determine whether that site can become the primary site of metabolism, and thus keep the overall metabolic rate of the drug essentially unchanged. The methods of the invention can also “profile” a compound for a particular ADMET/PK property. Such profile quantifies or bins each functional site on a compound based on the site's effect on a particular ADMET/PK property. For example, a given compound may include sites having varying degrees of lability. If a compound's lability profile suggests that 3 or more sites may have to be stabilized before the compound can be made sufficiently stable to exhibit a real improvement in metabolism rate, then the systems of this invention may drop that compound from further consideration. Hence, compound profiles can be used to filter potential analogs.

In a preferred embodiment of the invention, the analysis for redesigning a therapeutic compound is carried out by an integrated suite of software applications, each software application designed to analyze a subset of the ADMET/PK properties (e.g., one property) of the therapeutic compound and to suggest structural variants to improve upon the property. In a preferred embodiment the integrated suite contains software applications for analyzing at least two of the following properties: absorption (including but not limited to solubility, passive permeation of cell membranes, passive human and animal intestinal absorption, active transport across human and animal cell membranes), distribution (including but not limited to blood-brain-barrier penetration, human serum albumin binding, protein binding, receptor binding, drug transport across cell membranes), metabolism (including but not limited to compound binding affinity to metabolizing enzyme, rate of metabolism, regiolability, regiospecificity), excretion (including but not limited to hepatic and renal excretion), and toxicity (including but not limited to bioactivation of toxic metabolic intermediates, identification of structural features in the parent compound that are correlated with toxicity).

FIG. 3 is a block diagram that illustrates one preferred embodiment, 301, of the integrated suite of software applications that is used to redesign therapeutic compounds. The user/application interface 303, is used to communicate with the user, e.g., input from the user 315 and output to the user 317. It typically includes a graphic user interface by which the user can interact with the integrated suite. The user can input the name or structure of the therapeutic compound to be redesigned. The user can also indicate that the name or structure of the therapeutic compound should be received from elsewhere 319, or that output information should be sent elsewhere 321, e.g.. a database, another software application, or over the Internet.

This embodiment of the invention has a suite of software applications 302 for analyzing separate ADMET/PK properties. As shown, there is a separate software application for each of the following properties: absorption, distribution, metabolism, excretion and toxicity, as illustrated by the logic blocks 305, 307, 309, 311 and 313, respectively. The software for analyzing these properties and redesigning the therapeutic compound based on these properties can all be incorporated into one software application, or in some sub-combination (e.g., one software application can be used for 1 or 2 or more properties). Not all the software applications need to be used, though typically a compound will be analyzed for all these properties, even if the user does not envision redesigning the compound based on that property at the outset. After input of the therapeutic compound from the interface, analysis and redesign, the computational results is output back to the interface 303, the input and outputs for each application being indicated by the drawn arrows.

The computation systems of this invention may optionally include logic or functional blocks ancillary to the models in suite 302. This logic is collectively represented by block 330 in FIG. 3. It may perform various functions associated with the methodologies depicted above. For example, it may include a “structure generator” 333, which generates 3D structures or other appropriate structural representations of compounds to be analyzed by suite 302. Examples of suitable programs for this purpose were identified above. Ancillary logic 330 may also include an “analog selector” 335 that generates analogs or pharmacophore equivalents of the parent compound. These may serve as the candidates for ADMET/PK analysis and further consideration. Further, ancillary logic 330 may include a logic block for comparing the ADMET/PK properties of various compounds (parent compound and candidate replacements). See block 331. In this way, the system can automatically make meaningful comparisons and select potentially superior compounds. To this end, the system also include an ancillary activity prediction block 337 that predicts therapeutic activity from structural information. This may comprise any of a number of off the shelf or custom QSAR models for example.

### DETAILED DESCRIPTION OF THE PREFERRED EMBODIMENTS

Overview and Introduction

In the following detailed description of the present invention, numerous specific embodiments are set forth in order to provide a thorough understanding of the invention. However, as will be apparent to those skilled in the art, the present invention may be practiced without these specific details or by using alternate elements or processes. In other instances well known processes, procedures and components have not been described in detail so as not to unnecessarily obscure aspects of the present invention.

In this invention, therapeutic chemical compounds that have been designed and/or identified using conventional technology are now reevaluated with computational tools. These tools identify structurally related compounds that have at least one improved pharmacokinetic property. In most instances, the related compounds are chosen to preserve the therapeutic effectiveness of the parent compound. This approach may identify improved drugs that have fewer side effects, greater potency, etc. Typical candidates for use with this invention include marketed drugs having known deficiencies, withdrawn drugs that were withdrawn from the market because of a deficiency, and drugs in development that have been set aside because of discovered deficiencies. This invention allows the chemist to reengineer such compounds to provide new structures having modified features. The modified features are introduced to overcome or mitigate a known deficiency.

In many embodiments, the deficiency will be an ADMET/PK deficiency. As mentioned above, such deficiencies present significant costs to the industry and significant adversity to patients. Very often the deficiency is directly related to one or more structural features of the therapeutic compound. Some moieties can render toxic an a class of compounds that is otherwise safe. Other moieties can cause a compound to be metabolized too rapidly or too slowly.

The new compounds proposed by this invention will possess a structural modification of one or more features from the parent compound. The modification typically involves changing a limited portion of the parent compound's structure. In many cases, one reactive site or a small group of proximate sites on a compound impart an undesirable property. For example a particular site possessing a particular side group (e.g., an amine, an aldehyde, an ether, an amide, a carboxylic acid, a phenolic group, etc.) may impart the property. Or a combination of such features, either proximate to one another or separated in molecular space, may be responsible. Also, a scaffold or “interior” structure may be responsible for a particular orientation or conformation that imparts an undesirable ADMET property. In some cases, a ring or ring system will have a size, position, or constituent that causes the difficulty. For example, one or more hetero atoms in a ring may cause the difficulty.

In a simple example, a site with an unshielded ester group (e.g., a methyl or phenyl ester) or a vinyl group on a compound may be very labile and therefore cause the compound to metabolize too rapidly. Or a group of very strongly polar groups on a compound may cause the compound to be poorly absorbed. For example, the compound may include a carboxylic acid, amide, and/or amidine group that imparts very strong polarity inconsistent with good absorption. The methods and machines of this invention can identify such sites and/or propose alternative chemical structures for those sites to thereby suggest improved therapeutic compounds. For example, sites can be made less labile by substitution with appropriate fluorinated hydrocarbon groups or amide groups. And a compound that is too polar for good absorption can be improved by adding one or more perfluoro hydrocarbon groups, for example.

The methodology of this invention may be embodied in various combinations of method operations. Before or during the computational method, one or more therapeutic compounds susceptible to rescue must be flagged. The invention will propose one or more alternatives to these compounds (usually in the form of structural analogs or pharmacophore equivalents). These alternatives should preserve at least a significant degree of the therapeutic effectiveness of the parent compound and reduce one or more ADMET/PK deficiencies.

Some or all of the operations are typically implemented on a computational device (e.g., a computer operating under the control of software or firmware instructions). Example suitable software and hardware embodiments for practicing the software process are described below. In some methodologies one or more operations comprise non-computational experimental work. For example, the therapeutic effectiveness of a particular compound can in some instances only be evaluated by experimentation.

FIG. 1A is a flowchart that illustrates, from a very high-level, one suitable methodology, 101, for redesigning a therapeutic compound. Initially, in an operation 103, the therapeutic compound of interest is received by the software process. The therapeutic compound will typically be a legally-available drug, withdrawn drug, or lead compound that exhibits a therapeutic effect but is not ideal in terms of its ADMET/PK properties. As indicated, the general goal of the process described here is to improve the ADMET/PK properties while preserving or even improving the therapeutic effect of the compound.

The selected therapeutic compound is analyzed using the models described below to predict its ADMET/PK properties. See 105. Specifically, the models of this invention will quantify one or more ADMET/PK properties based upon a structural analysis of the compound. Since the therapeutic compound being analyzed was chosen in order to redesign its ADMET/PK properties, the actual ADMET/PK properties will often be known, and typically very precisely known, from empirical observations. However, sometimes the latent ADMET/PK deficiencies will be unknown until the compound is analyzed by models in accordance with this invention. Regardless, the properties should be quantified computationally so that they can be compared against predicted properties for analog compounds (potential rescuing analogs).

After analyzing the compound of interest, compounds that are structurally or pharmacophorically similar are identified. See 107. This identification process can be carried out in at least two manners. First, known compounds of a similar structure and similar therapeutic effect can be taken from any source, such as a biochemical database. Alternatively, compound structures can be generated de novo from the original structure of the chosen therapeutic compound. Various software tools can be used to perform this task, for example, MODDE and SIMCA available from Umetrics, Inc., of Umea, Sweden. Both of these alternatives also can be employed together.

For each candidate compound identified at 107, the computational system analyzes the chemical structure to predict the compound's ADMET/PK properties. See 109. This is preferably accomplished using the models employed to predict the parent compound's ADMET/PK properties. Again, these models quantify one or more ADMET/PK properties based upon a structural analysis of the compound.

At this point in the methodology, similar or identical computational techniques have been employed to predict the ADMET/PK properties of the parent and each candidate replacement. Hence a meaningful comparison can be made.

Finally, one or more of these compounds can be chosen as candidates for new drugs. See 111. Ideally, the chosen compounds have a predicted ADMET/PK property that is an improvement over a deficient ADMET/PK property of the parent compound. For example, the parent compound may be too toxic. The chosen compound(s) should have a predicted toxicity that is superior to that of the parent compound. Of course, the chosen compound need not be superior to the parent in every regard. For example, the

Once a drug has passed FDA trials and is offered on the market as an ethical pharmaceutical drug, the ADMET/PK properties may not be ideal. The drug may continue to be used, despite its ADMET/PK problems, or it may be withdrawn from use when a latent ADMET/PK problem is discovered. Another serious is that the drug may have adverse interactions with other compounds. One compound may induce or inhibit the metabolism, absorption or tissue distribution of another compound, thus altering its efficacy and sometimes increasing its toxicity to dangerous levels.

In view of the foregoing, it would be beneficial if ethically-used drugs, withdrawn drugs and lead compounds could be quickly and precisely redesigned to improve their ADMET/PK properties. Such a technique could “rescue” drugs that are undesirable because of their ADMET/PK properties, thus preventing the enormous cost of developing the drug in the first place from going to waste.

### SUMMARY OF THE INVENTION

The present invention relates generally to systems and methods for redesigning or “rescuing” ethical pharmaceutical drugs, withdrawn drugs, lead compounds and other compounds. More specifically, the invention relates to systems and methods for structurally redesigning such compounds in order to improve one or more of their ADMET/PK (absorption, distribution, metabolism, elimination, toxicity, i.e., pharmacokinetic) properties.

One aspect of the invention pertains to methods for redesigning a therapeutic chemical compound to improve at least one of its ADMET/PK properties, the method including the operations of identifying a therapeutic chemical compound that is a drug candidate, drug or withdrawn drug, analyzing the structure of the therapeutic chemical compound with a model that predicts one or more ADMET/PK properties of chemical compounds based upon their chemical structures, next, from an analysis of the therapeutic chemical compound with the model, identifying one or more structural features of the therapeutic chemical compound that cause the therapeutic chemical compound to possess an ADMET/PK property that can be improved, and specifying a modification for the one or more structural features to produce a modified chemical compound exhibiting an improvement in the one or more ADMET/PK properties over the therapeutic chemical compound, while substantially preserving a therapeutic effectiveness exhibited by the therapeutic chemical compound.

The therapeutic chemical compound may be a drug approved for sale by the U.S. Food and Drug Administration. It may have a demonstrated side effect attributable to one or more of its ADMET/PK properties. The model may predict a metabolic property of the therapeutic chemical compound, for example, in the form of separate lability values for individual reactive sites on the therapeutic chemical compound. The modification may involve one or more of the reactive sites to thereby speed up or slow down the rate at which the therapeutic chemical compound is metabolized. The model may predict binding affinity of the therapeutic compound to a P450 isozyme. The model may predict lability values for individual reactive sites on the therapeutic chemical compound by employing at least one of a quantum chemical model or an expression developed from a regression analysis performed on data associating reactivity with structural features.

The method may involve identifying one or more features of the therapeutic chemical compound that react most rapidly during metabolism, and comprises specifying one or more modifications of the one or more structural features. The model may predict how rapidly chemical compounds permeate cell layers. Some or all of the operations may be performed as software executing one or more processors. The therapeutic chemical compound may be received over the Internet. The therapeutic chemical may be selected from the group consisting of allopurinol, amiodarone, chloramphenicol, cipofloxacin, clarithomycin, diltiazem, dirithromycin, disulfiram, enoxacin, erythromycin, fluconazole, fluoxetine, fluvoxamine, isoniazid, itraconazole, ketoconazole, metronidazole, miconazole, MAOIs, nefazodone, omeprazole, trimethoprim/sulfamethoxazole, troleandomycin, verapamil, propoxyphene and quinidine.

A modification may reduce the therapeutic chemical compound's inhibitory effect on a CYP450 enzyme, example compounds being acetaminophen, amobarbital, aprobarbital, butabarbital, butalbital, carbamazepine, efavirenz, ethotoin, fosphenytoin, mephenytoin, mephobarbital, phenobarbital, phenytoin, primidone, rifabutin, rifampin, rifapentine and secobarbital. A modification may reduce the therapeutic chemical compound's toxicity example compounds being troglitazone, bromfenac, atorvastatin, zenarestat, trovafloxacin, cisapride, adefovir/dipivoxil, tolcapone, naproxen, ibuprofen and phentermine. The method may include analyzing the structure of the therapeutic chemical compound with multiple different models sequentially or in parallel, each model predicting one or more ADMET/PK properties of chemical compounds.

In predicting absorption, the absorption property may have at least one component selected from the following: solubility, passive permeation of cell membranes, passive intestinal absorption, active transport across cell membranes. In predicting metabolism, the metabolism property may have at least one component selected from the following: binding affinity to metabolizing enzyme, rate of metabolism and regiolability. In predicting excretion, the excretion property may have at least one component selected from the following: hepatic excretion and renal excretion. In predicting toxicity, the toxicity property may have at least one component selected from the following: bioactivation of toxic metabolic intermediates, and structural features in the parent compound that are correlated with toxicity. In predicting distribution, the distribution property may have at least one component selected from the following: blood-brain-barrier penetration, human serum albumin binding, protein binding, receptor binding and transport across cell membranes. The methods may also include simultaneously displaying the analyzed ADMET/PK properties of a library of compounds.

Another aspect of the invention pertains to an apparatus for analyzing therapeutic chemical compounds in order to redesign the therapeutic chemical compounds, the apparatus including an interface for receiving information, including the identity of chemical compounds, from at least one external source, a memory device for storing, at least temporarily, chemical structural information of a therapeutic chemical compound that is a drug candidate, drug or withdrawn drug and instructions for analyzing one or more ADMET/PK properties of the therapeutic chemical compound, and one or more processors designed or configured to identify one or more structural features of the therapeutic chemical compound that cause the therapeutic chemical compound to possess an ADMET/PK property that can be improved, and specify a modification for the one or more structural features to produce a modified chemical compound exhibiting an improvement in the one or more ADMET/PK properties over the therapeutic chemical compound, while substantially preserving a therapeutic effectiveness exhibited by the therapeutic chemical compound.

The one or more processors may be configured to perform some of these operations executing said instructions stored in memory and may be configured with a software application or an integrated suite of software tools for analyzing the therapeutic chemical compounds. The interface may allow connection to the Internet so that the therapeutic chemical compound can be identified by receiving the compound's identity over the Internet. The apparatus may also include a software program for providing a structural representation of the therapeutic chemical compound from the identity of the therapeutic chemical compound.

What is claimed is:

1. A method of redesigning a therapeutic chemical compound to improve at least one of its ADMET/PK properties, the method comprising:
(a) identifying a therapeutic chemical compound that is a drug or withdrawn drug;
(b) analyzing the structure of the therapeutic chemical compound with a model that predicts one or more ADMET/PK properties of chemical compounds based upon their chemical structures;
(c) from an analysis of the therapeutic chemical compound with the model, identifying one or more structural features of the therapeutic chemical compound that cause the therapeutic chemical compound to possess an ADMET/PK property that can be improved; and
(d) specifying a modification for the one or more structural features identified in (c) to produce a modified chemical compound exhibiting an improvement in the one or more ADMET/PK properties over the therapeutic chemical compound, while substantially preserving a therapeutic effectiveness exhibited by the therapeutic chemical compound.

2. The method of claim 1, wherein the therapeutic chemical compound is a drug approved for sale by the U.S. Food and Drug Administration.

3. The method of claim 1, wherein the therapeutic chemical compound has a demonstrated side effect attributable to one or more of its ADMET/PK properties.

4. The method of claim 1, wherein the model predicts a metabolic property of the therapeutic chemical compound.

5. The method of claim 4, wherein the model predicts the metabolic property in the form of separate lability values for individual reactive sites on the therapeutic chemical compound.

6. The method of claim 5, wherein the modification specified at (d) is accomplished by modifying one or more of the reactive sites to thereby speed up or slow down the rate at which the therapeutic chemical compound is metabolized.

7. The method of claim 1, wherein the model predicts binding affinity of the therapeutic compound to a P450 isozyme.

8. The method of claim 4, wherein the model predicts lability values for individual reactive sites on the therapeutic chemical compound by employing at least one of a quantum chemical model or an expression developed from a regression analysis performed on data associating reactivity with structural features.

9. The method of claim 1, wherein (c) comprises identifying one or more features of the therapeutic chemical compound that react most rapidly during metabolism, and wherein (d) comprises specifying one or more modifications of the one or more structural features.

10. The method of claim 1, wherein the model predicts how rapidly chemical compounds permeate cell layers.

11. The method of claim 1, wherein (a) through (d) are performed as software executing one or more processors.

12. The method of claim 1, wherein (b) through (d) are performed as software executing one or more processors.

13. The method of claim 1, wherein the therapeutic chemical compound is identified by receiving the compound's identity over the Internet.

14. The method of claim 1, wherein the therapeutic chemical compound is selected from the group consisting of allopurinol, amiodarone, chloramphenicol, cipofloxacin, clarithomycin, diltiazem, dirithromycin, disulfiram, enoxacin, erythromycin, fluconazole, fluoxetine, fluvoxamine, isoniazid, itraconazole, ketoconazole, metronidazole, miconazole, MAOIs, nefazodone, omeprazole, trimethoprim/sulfamethoxazole, troleandomycin, verapamil, propoxyphene and quinidine.

15. The method of claim 1, wherein the modification reduces the therapeutic chemical compound's inhibitory effect on a CYP450 enzyme.

16. The method of claim 15, wherein the therapeutic chemical compound is selected from the group consisting of amiodarone, chloramphenicol, cimetidine, diltiazem, fluoxetine, fluvoxamine, indinavir, itraconazole, ketoconazole, mibefradil, paroxetine, propafenone, quinidine, ritonavir, sertraline and troleandomycin.

17. The method of claim 1, wherein the modification reduces the therapeutic chemical compound's induction effect on a CYP450 enzyme.

18. The method of claim 17, wherein the therapeutic chemical compound is selected from the group consisting of acetaminophen, amobarbital, aprobarbital, butabarbital, butalbital, carbamazepine, efavirenz, ethotoin, fosphenytoin, mephenytoin, mephobarbital, phenobarbital, phenytoin, primidone, rifabutin, rifampin, rifapentine and secobarbital.

19. The method of claim 1, wherein the modification reduces the therapeutic chemical compound's toxicity.

20. The method of claim 19, wherein the therapeutic chemical compound is selected from the group consisting of troglitazone, bromfenac, atorvastatin, zenarestat, trovafloxacin, cisapride, adefovir/dipivoxil, tolcapone, naproxen, ibuprofen and phentermine.

21. The method of claim 1, wherein (b) comprises analyzing the structure of the therapeutic chemical compound with multiple different models sequentially or in parallel, each model predicting one or more ADMET/PK properties of chemical compounds.

22. The method of claim 1, further comprising predicting the absorption property, wherein the absorption property has at least one component selected from the following: solubility, passive permeation of cell membranes, passive intestinal absorption, active transport across cell membranes.

23. The method of claim 1, further comprising predicting the metabolism property, wherein the metabolism property has at least one component selected from the following: binding affinity to metabolizing enzyme, rate of metabolism and regiolability.

24. The method of claim 1 further comprising predicting the excretion property, wherein the excretion property has at least one component selected from the following: hepatic excretion and renal excretion.

25. The method of claim 1, further comprising predicting the toxicity property, wherein the toxicity property has at least one component selected from the following: bioactivation of toxic metabolic intermediates, and structural features in the parent compound that are correlated with toxicity.

26. The method of claim 1, further comprising predicting the distribution property, wherein the distrigbution property has at least one component selected from the following: blood-brain-barrier penetration, human serum albumin binding, protein binking, receptor binding and transport across cell membranes.

27. The method of claim 1 further comprising simultaenously displaying the analyzed ADMET/PK properties of a library of compounds.

28. A computer-program product comprising a computer-readable medium and program instructions provided via the computer-readable medium, the program instructions comprising instructions for redesigning a therapentic chemical compound to improve at least one of its ADMET/PK properties, the instructions specifying:
(a) identifying a therapeutic chemical compound that is a drug or withdrawn drug;
(b) analyzing the structure of the therapeutic chemical compound witha model that predicts one or more ADMET/PK properties of chemical compounds based upon their chemical structures;
(c) from an analysis of the therapeutic chemeical compound with the model, identifying one or more structural features of the therapeutic chemical compound that cause the therapeutic chemical compound to possess an ADMET/PK property that can be improved; and
(d) specifying a modification for the one or more structural features identified in (c) to produce a modified chemical compound exhibiting an improvement in the one or more ADMET/PK properties over the therapeutic chemical compound, while substantially preserving a therapeutic effectiveness exhibited by the therapeutic chemical compound.

29. The computer-program product of claim 28, wherein the therapeutic chemical compound is a drug approved for sake by the U.S. Food and Drue Administration.

30. The computer-program product of claim 28, wherein the therapeutic chemical compound has a demonstrated side effect attributable to one or more of its ADMET/PK properties.

31. The computer-program product of claim 28, wherein the therapeutic chemical compound has a demonstrated side effect attributable to one or more of its ADMET/PK properties.

32. The computer-program product of claim 28, wherein the modification specified at (d) is accomplished by modifying one or more of the reactive sites to thereby speed up or slow down the rate at which the therapeutic chemical compound is metabolized.

33. The computer-program product of claim 28, further comprising instructions for predicting lability values for individual reactive sites on the therapeutic chemical compound by employing at least one of a quantum chemical model or an expression edveloped from a regression analysis performed on data associating reactivity with structural features.

34. The computer-program product of claim 28, further comprising instructions for producting wherein (c) comprises identifying one or more features of the therapeutic chemical compound that react most rapidly during metabolism, and wherein (d) comprises specifying one or more modifications of the one or more structural features.

35. The computer-program product of claim 28, further comprising instructions for predicting how rapidly chemical compounds permeate cell layers.

36. The computer-progrm product of claim 28, wherein the modification reduces the therapeutic chemical compound's inhibitory effect on a CYP450 enzyme.

37. The computer-progrm product of claim 28, wherein the modification reduces the therapeutic chemical compound's induction effect on a CYP450 enzyme.

38. The computer-progrm product of claim 28, wherein the modification reduces the therapeutic chemical compound's toxicity.

39. The computer-program product of claim 28, further comprising instructions for predicting the absorption property, wherein the absorption property has at least one component selected from the following: solubility, passive permcation of cell membranes, passice intestinal absorption, active transport across cell membranes.

40. The computer-program product of claim 28, further comprising instructions for predicting the absorption property, wherein the metabolism property has at least one component selected from the following: binking affinity to metabolizing anzyme, rate of metabolism and regiolability.

41. The computer-program product of claim 28, furthe rcomprising instructions for predicting the excretion property, wherein the metabolism property has at least one component selected from the following: hepatic excetion and renal excretion.

42. The computer-program product of claim 28, further comprising instructions for predicting the toxicity property, wherein the toxicity property has at least one component selected rom the following: bioactivation of toxic metabolic intermediates, and structural features in the parent compound that are correlated with toxicity.

43. The computer-program product of claim 28, further comprising instructions for preduicting the distribution property, wherein the distribution property has at least one component selected from the following: blood-brain-barrier penetration, human serum albumin binding, protein binding, receptor binding and transport across cell membranes.

44. An apparatus for analyzing therapeutic chemical compounds in order ot redesign the therapeutic chemical compounds, the apparatue comprising:
(a) an interface for receiving information, including the identity of chemical compounds, form at least one external source;
(b) a memory device for storing, at least temporarily, (i) chemical structural information of a therapeutic chemical compound that is a drug cndidate, drug or withdrawn drug and (ii) instructions for analyzing one or more ADMET/PK propertics of the therapeutic chemical compound; and
(c) on or more processors designed or configured to
(i) identify one or more structural features of the therapeutic chemical compound that cause the therapeutic chemical compound to possess an ADMET/PK property that can be improved, and
(ii) specify a modification for the one or more structural features to produce a modified chemical compound exhibiting an improvement in the one or more ADMET/PK properties over the therapeutic chemical compound, while substantially preserving a therapeutic effectiveness exhibited by the therapeutic chemical compound.

45. The apparatus of claim 44, wherein the one or more processors are configured to perform (i) and (ii) by executing said instructions stored in memory.

46. The apparatus of claim 44, wherein in the one or more processors are configured with a software application or an integrated suite of software tools for analyzing the therapeutic chemical compounds.

47. The apparatus of claim 44, wherein the interface allows connection to the Internet so that the therapeutic chemical compound can be identified by receiving the compound's identity over the Internet.

48. The apparatus of claim 44, further comprising a software program for providing a structural representation of the therapeutic chemical compound from the identity of the therapeutic chemical compound.

49. The apparatus of claim 44, wherein the one or more processors are designed or configured to predict a metabolic property of the therapeutic chemical compound.

50. The apparatus of claim 49, wherein the one or more processors are designed or configured to predict the metabolic property in the form of separate lability values for individual reactive sites on the therapeutic chemical compound.

51. The apparatus of claim 50, wherein the one or more processors are designed or configured to specify a modification for the one or more structural features by modifying one or more of the reactive sites to thereby speed up or slow down the rate at which the therapeutic chemical compound is metabolized.

52. The apparatus of claim 50, wherein the one or more processors are designed or configured to predict lability values for individual reactive sites on the therapeutic chemical compound by employing at least one of a quantum mechanical model or an expression developed from a regression analysis performed on data associating reactivity with structural features.

53. The apparatus of claim 44, wherein the one or more processors are designed or configured to predict how rapidly chemical compounds are absorbed into the blood stream.

54. A method of analyzing a therapeutic chemical compound to redesign the therapeutic chemical compound, the method comprising:
(a) identifying a therapeutic chemical compound that is a drug candidate, drug or withdrawn drug;
(b) utilizing a software application or integrated suite of software applications to analyze the therapeutic chemical compound, wherein the software application or integrated suite of software applications can analyze at least two of the following properties of the therapeutic chemical compound: absorption, metabolism, distribution and toxicity; and
(c) specifying a modification to the therapeutic compound in order to modify at least one of the following properties of the therapeutic compound: absorption, metabolism, distribution, excretion, and toxicity.

55. The method of claim 54, wherein the therapeutic chemical compound is a drug approved for sale by the U.S. Food and Drug Administration.

56. The method of claim 54, wherein the therapeutic chemical compound has a demonstrated side effect attributable to one or more of its ADMET/PK properties.

57. The method of claim 54, wherein the therapeutic chemical compound is identified by receiving the compound's identity over the Internet.

58. The method of claim 54, wherein the therapeutic chemical compound is identified by receiving the compound's identity from a user's input.

59. The method of claim 54, wherein (a) through (c) are performed as software executing on one or more processors.

60. The method of claim 54, wherein the therapeutic chemical compound is selected from the group consisting of allopurinol, amiodarone, chloramphenicol, cipofloxacin, clarithomycin, diltiazem, dirithromycin, disulfiram, enoxacin, erythromycin, fluconazole, fluoxetine, fluvoxamine, isoniazid, itraconazole, ketoconazole, metronidazole, miconazole, MAOIs, nefazodone, omeprazole, trimethoprim/sulfamethoxazole, troleandomycin, verapamil, propoxyphene and quinidine.

61. The method of claim 54, wherein the modification reduces the therapeutic chemical compound's inhibitory effect on a CYP450 enzyme.

62. The method of claim 61, wherein the therapeutic chemical compound is selected from the group consisting of amiodarone, chloramphenicol, cimetidine, diltiazem, fluoxetine, fluvoxamine, indinavir, itraconazole, ketoconazole, mibefradil, paroxetine, propafenone, quinidine, ritonavir, sertraline and troleandomycin.

63. The method of claim 54, wherein the modification reduces the therapeutic chemical compound's induction effect on a CYP450 enzyme.

64. The method of claim 63, wherein the therapeutic chemical compound is selected from the group consisting of acetaminophen, amobarbital, aprobarbital, butabarbital, butalbital, carbamazepine, efavirenz, ethotoin, fosphenytoin, mephenytoin, mephobarbital, phenobarbital, phenytoin, primidone, rifabutin, rifampin, rifapentine and secobarbital.

65. The method of claim 54, wherein the modification reduces the therapeutic chemical compound's toxicity.

66. The method of claim 65, wherein the therapeutic chemical compound is selected from the group consisting of troglitazone, bromfenac, atorvastatin, zenarestat, trovafloxacin, cisapride, adefovir/dipivoxil, tolcapone, naproxen, ibuprofen and phentermine.

67. The method of claim 54, wherein the software application or integrated suite of software applications analyzes a metabolic property of the therapeutic chemical compound.

68. The method of claim 67, wherein the software application or integrated suite of software applications analyzes the metabolic property in the form of separate lability values for individual reactive sites on the therapeutic chemical compound.

69. The method of claim 68, wherein the modification specified at (c) is accomplished by modifying one or more of the reactive sites to thereby speed up or slow down the rate at which the therapeutic chemical compound is metabolized.

70. The method of claim 69, wherein the software application or integrated suite of software applications analyzes lability values for individual reactive sites on the therapeutic chemical compound by employing at least one of a quantum mechanical model or an expression developed from a regression analysis performed on data associating reactivity with structural features.

71. The method of claim 70, wherein the software application or integrated suite of software applications analyzes how rapidly chemical compounds are absorbed into the blood stream.

72. A computer-program product comprising a computer-readable medium and program instructions provided via the computer-readable medium, the program instructions comprising instructions for analyzing a therapeutic compound to redesign the therapeutic chemical compound, the computer-program product comprising instructions for:
(a) identifying a therapeutic chemical compound that is a drug candidate, drug or withdrawn drug;
(b) utilizing a software application or integrated suite of software applications to analyze the therapeutic chemical compound, wherein the software application or integrated suite of software applications can analyze at least two of the following properties of the therapeutic chemical compound: absorption, metabolism, distribution, and toxicity; and
(c) specifying a modification to the therapeutic compound in order to modify at least one of the following properties of the therapeutic compound: absorption, metabolism, distribution, excretion, and toxicity.

73. The computer-program product of claim 72, wherein the therapeutic chemical compound is a drug approved for sale by the U.S. Food and Drug Administration.

74. The computer-program product of claim 72, wherein the therapeutic chemical compound has a demonstrated side effect attributable to one or more of its ADMET/PK properties.

75. The computer-program product of claim 72, wherein the therapeutic chemical compound is identified by receiving the compound's identity over the Internet.

76. The computer-program product of claim 72, wherein the therapeutic chemical compound is identified by receiving the compound's identity from a user's input.

77. The computer-program product of claim 72, wherein the therapeutic chemical compound is selected from the group consisting of allopurinol, amiodarone, chloramphenicol, cipofloxacin, clarithomycin, diltiazem, dirithromycin, disulfiram, enoxacin, erythromycin, fluconazole, fluoxetine, fluvoxamine, isoniazid, itraconazole, ketoconazole, metronidazole, miconazole, MAOIs, nefazodone, omeprazole, trimethoprim/sulfamethoxazole, troleandomycin, verapamil, propoxyphene and quinidine.

78. The computer-program product of claim 72, wherein the modification reduces the therapeutic chemical compound's inhibitory effect on a CYP450 enzyme.

79. The computer-program product of claim 72, wherein the modification reduces the therapeutic chemical compound's induction effect on a CYP450 enzyme.

80. The computer-program product of claim 72, wherein the modification reduces the therapeutic chemical compound's toxicity.

81. An apparatus for analyzing therapeutic chemical compounds to redesign the therapeutic chemical compounds, the apparatus comprising:
(a) an interface for receiving information, including the identity of chemical compounds, from at least one external source;
(b) a memory device for storing, at least temporarily, (i) chemical structural information of a therapeutic chemical compound that is a drug candidate, drug or withdrawn drug and (ii) instructions for analyzing at least two of the following properties of the therapeutic chemical compound: absorption, metabolism, distribution and toxicity; and
(c) one or more processors designed or configured to
(i) analyze the therapeutic chemical compound stored in memory by evaluating at least two of the following properties of the therapeutic chemical compound: absorption, metabolism, distribution and toxicity, and
(ii) specify a modification to the therapeutic compound in order to modify at least one of the following properties of the therapeutic compound: absorption, metabolism, distribution and toxicity.

82. The apparatus of claim 81, wherein in the one or more processors are configured with a software application or an integrated suite of software tools for analyzing the therapeutic chemical compounds.

83. The apparatus of claim 81, wherein the interface allows connection to the Internet so that the therapeutic chemical compound can be identified by receiving the compound's identity over the Internet.

84. The apparatus of claim 81, further comprising a software program for providing a structural representation of the therapeutic chemical compound from the identity of the therapeutic chemical compound.

85. The apparatus of claim 81, wherein the one or more processors are designed or configured to evaluate a metabolic property of the therapeutic chemical compound.

86. The apparatus of claim 81, wherein the one or more processors are designed or configured to evaluate the metabolic property in the form of separate lability values for individual reactive sites on the therapeutic chemical compound.

87. The apparatus of claim 86, wherein the one or more processors are designed or configured to specify a modification for the one or more structural features by modifying one or more of the reactive sites to thereby speed up or slow down the rate at which the therapeutic chemical compound is metabolized.

88. The apparatus of claim 86, wherein the one or more processors are designed or configured to predict how rapidly chemical compounds are absorbed into the blood stream.

89. The method of claim 1, wherein the therapeutic chemical compound has its therapeutic properties affected by a drug-drug interaction with at least one of a metabolism inducer or a metabolism inhibitor.

90. The method of claim 1, wherein the therapeutic chemical compound is selected from the group consisting of acenocoumarol, acetaminophen, acetophenazine, alprazolam, alprazolam, amiodarone, amiodarone, amitriptyline, amitriptyline, betamethasone, amoxapine, bupropion, astemizole, buspirone, atorvastatin, carbamazepine, bepridil, chlordiazepoxide, buspirone, clarithromycin, carbamazepine, clomipramine, cerivastatin, clonazepam, chlordiazepoxide, clorazepate, chloroquine, clozapine, chlorpromazine, corticotropin, chlorzoxazone, cortisone, cisapride, cosyntropin, clarithromycin, cyclosporine, clomipramine, delavirdine, clonazepam, desipramine, clorazepate, detromethorphan, clozapine, dexamethasone, codeine, diazepam, cyclosporine, disopyramide, desipramine, doxepin, diazepam, doxycycline, dihydroergotamine, erythromycin, doxepin, estazolam, encainide, felodipine, ergotamine, fludrocortisone, estazolam, flurazepam, felodipine, halazepam, fentanyl, haloperidol, flecainide, hydrocortisone, fluphenazine, imipramine, flurazepam, indinavir, fluvastatin, itraconazole, halazepam, ketoconazole, hydrocortisone, mephenytoin, imipramine, methadone, indinavir, methylprednisolone, lidocaine, miconazole, lovastatin, midazolam, mephenytoin, nelfinavir, mesoridazine, nifedipine, methotrimeprazine, nisoldipine, methylprednisolone, nortriptyline, metoprolol, oral contraceptives, midazolam, phenobarbital, nifedipine, phenytoin, nisoldipine,, prednisolone, nortriptyline, prednisone, omeprazole, quazepam, oral contraceptives, quinidine, perphenazine, quinine, phenprocoumon, ritonavir, phenytoin, tacrolimus, pimozide, tamoxifen, piroxicam, toremifene, pravastatin, triamcinolone, prazapam, triazolam, prednisolone, trimipramine, prednisone, troleandomycin, prochlorperazine, verapamil, promazine, warfarin, promethazine, zolpidem, propafenone, propiomazine, propoxyphene, propranolol, protriptyline, quazepam, quinidine, rifabutin, rifampin, rifapentine, ritonavir, saquinavir, sildenafil, simvastatin, tacrolimus, terfenadine, thiethylperazine, thioridazine, timolol, tolbutamide, triazolam, trifluoperazine, triflupromazine, trimipramine, valproic acid, vinblastine, vincristine, warfarin, and zolpidem.

91. The method of claim 54, wherein the therapeutic chemical compound has its therapeutic properties affected by a drug-drug interaction with at least one of a metabolism inducer or a metabolism inhibitor.

92. The method of claim 54, wherein the therapeutic chemical compound is selected from the group consisting of acenocoumarol, acetaminophen, acetophenazine, alprazolam, alprazolam, amiodarone, amiodarone, amitriptyline, amitriptyline, betamethasone, amoxapine, bupropion, astemizole, buspirone, atorvastatin, carbamazepine, bepridil, chlordiazepoxide, buspirone, clarithromycin, carbamazepine, clomipramine, cerivastatin, clonazepam, chlordiazepoxide, clorazepate, chloroquine, clozapine, chlorpromazine, corticotropin, chlorzoxazone, cortisone, cisapride, cosyntropin, clarithromycin, cyclosporine, clomipramine, delavirdine, clonazepam, desipramine, clorazepate, detromethorphan, clozapine, dexamethasone, codeine, diazepam, cyclosporine, disopyramide, desipramine, doxepin, diazepam, doxycycline, dihydroergotamine, erythromycin, doxepin, estazolam, encainide, felodipine, ergotamine, fludrocortisone, estazolam, flurazepam, felodipine, halazepam, fentanyl, haloperidol, flecainide, hydrocortisone, fluphenazine, imipramine, flurazepam, indinavir, fluvastatin, itraconazole, halazepam, ketoconazole, hydrocortisone, mephenytoin, imipramine, methadone, indinavir, methylprednisolone, lidocaine, miconazole, lovastatin, midazolam, mephenytoin, nelfinavir, mesoridazine, nifedipine, methotrimeprazine, nisoldipine, methylprednisolone, nortriptyline, metoprolol, oral contraceptives, midazolam, phenobarbital, nifedipine, phenytoin, nisoldipine,, prednisolone, nortriptyline, prednisone, omeprazole, quazepam, oral contraceptives, quinidine, perphenazine, quinine, phenprocoumon, ritonavir, phenytoin, tacrolimus, pimozide, tamoxifen, piroxicam, toremifene, pravastatin, triamcinolone, prazapam, triazolam, prednisolone, trimipramine, prednisone, troleandomycin, prochlorperazine, verapamil, promazine, warfarin, promethazine, zolpidem, propafenone, propiomazine, propoxyphene, propranolol, protriptyline, quazepam, quinidine, rifabutin, rifampin, rifapentine, ritonavir, saquinavir, sildenafil, simvastatin, tacrolimus, terfenadine, thiethylperazine, thioridazine, timolol, tolbutamide, triazolam, trifluoperazine, triflupromazine, trimipramine, valproic acid, vinblastine, vincristine, warfarin, and zolpidem.
